(19) United States
(12) Patent Application Publication
(10) Pub. No.: US 2015/0218164 A1
(43) Pub. Date: Aug. 6, 2015

(54) SUBSTITUTED NAPHTHYRIDINEDIONE DERIVATIVES AS HIV INTEGRASE INHIBITORS

(71) Applicant: MERCK SHARP & DOHME CORP., Rahway, NJ (US)

(72) Inventors: Paul J. Coleman, Harleysville, PA (US); Mark Embrey, Harleysville, PA (US); Timothy J. Hartingh, Blue Bell, PA (US); David Powell, Lansdale, PA (US); Izzat T. Raheem, Doylestown, PA (US); Ronald K. Chang, West Point, PA (US); John Schreier, Harleysville, PA (US); John Sisko, Lansdale, PA (US); Thomas G. Steele, Schwenksville, PA (US); Abbas M. Walji, West Point, PA (US)

(21) Appl. No.: 14/416,923

(22) PCT Filed: Jul. 22, 2013
(86) PCT No.: PCT/US13/51494

§ 371 Date: Jan. 23, 2015

Related U.S. Application Data
(60) Provisional application No. 61/675,787, filed on Jul. 25, 2012.

(51) Int. Cl. C07D 471/14; C07F 9/6561; A61K 31/4375; A61K 31/513; A61K 31/505; A61K 31/427; A61K 31/5365; A61K 31/635; A61K 31/4418; A61K 31/47; A61K 31/675; A61K 31/52

(52) U.S. Cl. CPC C07D 471/14; A61K 31/675; C07F 9/65615; A61K 31/4375; A61K 31/513; A61K 31/52; A61K 31/427; A61K 31/5365; A61K 31/635; A61K 31/4418; A61K 31/47; A61K 31/505

(57) ABSTRACT

The present invention relates to Substituted Naphthyridinedione Derivatives and pharmaceutically acceptable salts thereof. The present invention also relates to compositions comprising at least one Substituted Naphthyridinedione Derivative, and methods of using the Substituted Naphthyridinedione Derivatives for treating or preventing HIV infection in a subject.

### FIELD OF THE INVENTION

The present invention relates to Substituted Naphthyridinedione Derivatives, compositions comprising at least one Substituted Naphthyridinedione Derivative, and methods of using the Substituted Naphthyridinedione Derivatives for treating or preventing HIV infection in a subject.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277(1985)] Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication.

The following references are of interest as background:

International Publication Nos. WO 11/045330 and WO 11/121105 disclose macrocyclic compounds having HIV integrase inhibitory activity.

Kinzel et al., Tet. Letters 2007, 48(37): pp. 6552-6555 discloses the synthesis of tetrahydropyridopyrimidones as a scaffold for HIV-1 integrase inhibitors.

Ferrara et al., Tet. Letters 2007, 48(37), pp. 8379-8382 discloses the synthesis of a hexahydropyrimido[1,2-a]azepine-2-carboxamide derivative useful as an HIV integrase inhibitor.

Muraglia et al., J. Med. Chem. 2008, 51: 861-874 discloses the design and synthesis of bicyclic pyrimidinones as potent and orally bioavailable HIV-1 integrase inhibitors.

US2004/229909 discloses certain compounds having integrase inhibitory activity.

U.S. Pat. No. 7,232,819 and US 2007/0083045 disclose certain 5,6-dihydroxypyrimidine-4-carboxamides as HIV integrase inhibitors.

U.S. Pat. No. 7,169,780, U.S. Pat. No. 7,217,713, and US 2007/0123524 disclose certain N-substituted 5-hydroxy-6-oxo-1,6-dihydropyrimidine-4-carboxamides as HIV integrase inhibitors.

U.S. Pat. No. 7,279,487 discloses certain hydroxynaphthyridinone carboxamides that are useful as HIV integrase inhibitors.

U.S. Pat. No. 7,135,467 and U.S. Pat. No. 7,037,908 disclose certain pyrimidine carboxamides that are useful as HIV integrase inhibitors.

U.S. Pat. No. 7,211,572 discloses certain nitrogenous condensed ring compounds that are HIV integrase inhibitors.

U.S. Pat. No. 7,414,045 discloses certain tetrahydro-4H-pyrido[1,2-a]pyrimidine carboxamides, hexahydropyrimido[1,2-a]azepine carboxamides, and related compounds that are useful as HIV integrase inhibitors.

WO 2006/103399 discloses certain tetrahydro-4H-pyrimidooxazepine carboaxmides, tetrahydropyrazinopyrimidine carboxamides, hexahydropyrimidodiazepine carboxamides, and related compounds that are useful as HIV integrase inhibitors.

US 2007/0142635 discloses processes for preparing hexahydropyrimido[1,2-a]azepine-2-carboxylates and related compounds.

US 2007/0149556 discloses certain hydroxypyrimidinone derivatives having HIV integrase inhibitory activity.

Various pyrimidinone compounds useful as HIV integrase inhibitors are also disclosed in U.S. Pat. No. 7,115,601, U.S. Pat. No. 7,157,447, U.S. Pat. No. 7,173,022, U.S. Pat. No. 7,176,196, U.S. Pat. No. 7,192,948, U.S. Pat. No. 7,273,859, and U.S. Pat. No. 7,419,969.

US 2007/0111984 discloses a series of bicyclic pyrimidinone compounds useful as HIV integrase inhibitors.

US 2006/0276466, US 2007/0049606, US 2007/0111985, US 2007/0112190, US 2007/0281917, US 2008/0004265 each disclose a series of bicyclic pyrimidinone compounds useful as HIV integrase inhibitors.

### SUMMARY OF THE INVENTION

In one aspect, the present invention provides Compounds of Formula (I):

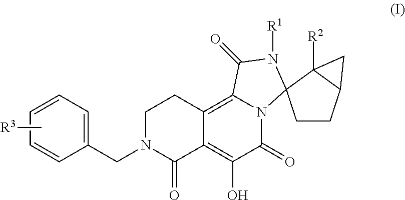

and pharmaceutically acceptable salts and prodrugs thereof,
wherein:

R¹ is H or C₁-C₄ alkyl, wherein said C₁-C₄ alkyl group can be optionally substituted with up to two groups, each independently selected from —OH, F, —OP(O)(OH)₂ and —OC(O)(C₁-C₆ alkyl), wherein the C₁-C₆ alkyl moiety of said —OC(O)(C₁-C₆ alkyl) substituent group can be optionally substituted with up to 2 groups, each independently selected from —N(R⁵)₂, —C(O)N(R⁵)₂ and —S—(C₁-C₃ alkyl);

R² is H or —(C₁-C₃ alkylene)-R⁴;

R³ represents up to 2 optional phenyl ring substituents, which are each independently selected from halo;

R⁴ is selected from —OH, —SH, —S—(C₁-C₃ alkyl), —SO₂(C₁-C₃ alkyl) and —OP(O)(OH)₂; and

each occurrence of R⁵ is H or C₁-C₆ alkyl, or two R⁵ groups that are attached to the same nitrogen atom, together with the common nitrogen atom to which they are attached, join to form a 4 to 7-membered heterocycloalkyl group.

The Compounds of Formula (I) (also referred to herein as the “Substituted Naphthyridinedione Derivatives”) and pharmaceutically acceptable salts thereof can be useful, for example, for inhibiting HIV viral replication or replicon activity, and for treating or preventing HIV infection in a subject. Without being bound by any specific theory, it is believed that the Substituted Naphthyridinedione Derivatives inhibit HIV viral replication by inhibiting HIV Integrase.

Accordingly, the present invention provides methods for treating or preventing HIV infection in a subject, comprising administering to the subject an effective amount of at least one Substituted Naphthyridinedione Derivative.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to Substituted Naphthyridinedione Derivatives, compositions comprising at least one Substituted Naphthyridinedione Derivative, and methods of using the Substituted Naphthyridinedione Derivatives for inhibiting HIV integrase, inhibiting HIV viral replication or for treating or preventing HIV infection in a subject.

### DEFINITIONS AND ABBREVIATIONS

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . . .

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

The term “effective amount” as used herein, refers to an amount of Substituted Naphthyridinedione Derivative and/or an additional therapeutic agent, or a composition thereof that is effective in producing the desired therapeutic, ameliorative, inhibitory or preventative effect when administered to a subject suffering from HIV infection or AIDS. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HIV viral infection or AIDS, refers to reducing the likelihood or severity of HIV infection or AIDS.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group has from about 3 to about 5 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms. The term “C₃-C₅ alkylene” refers to an alkylene group having from 3 to 5 carbon atoms.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH═CHCH₂—, —CH₂CH═CH—, —CH₂CH═CHCH₂—, —CH═CHCH₂CH₂—, —CH₂CH₂CH═CH— and —CH(CH₃)CH═CH—. In one embodiment, an alkenylene group has from 2 to about 6 carbon atoms. In another embodiment, an alkenylene group has from about 3 to about 5 carbon atoms. In another embodiment, an alkenylene group is branched. In another embodiment, an alkenylene group is linear. The term “C₂-C₆ alkylene” refers to an alkenylene group having from 2 to 6 carbon atoms. The term “C₃-C₅ alkenylene” refers to an alkenylene group having from 3 to 5 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

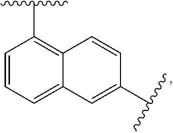 [SMILES: *c1ccc2c(*)cccc2c1]

is understood to represent both:

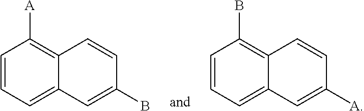

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

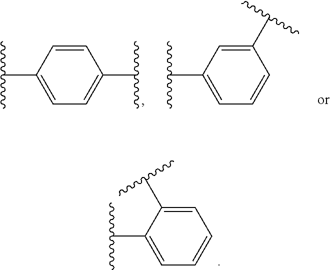

Unless otherwise indicated, an arylene group is unsubstituted.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 7-membered cycloalkyl” refers to a cycloalkyl group having from 3 to 7 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to cyclobutanoyl:

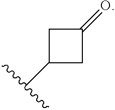 [SMILES: *C1CC(=O)C1]

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms have been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered monocyclic heteroaryl. In another embodiment, a heteroaryl group comprises a 5- to 6-membered monocyclic heteroaryl group fused to a benzene ring. Unless otherwise indicated, a heteroaryl group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone and the like, and all isomers thereof

A ring carbon atom of a heterocycloalkyl group may be functionalized as a carbonyl group. An illustrative example of such a heterocycloalkyl group is:

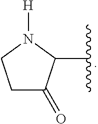 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic heterocycloalkyl” refers to a monocyclic heterocycloalkyl group having from 3 to 6 ring atoms. The term “4 to 7-membered monocyclic heterocycloalkyl” refers to a monocyclic heterocycloalkyl group having from 4 to 7 ring atoms. The term “7 to 11-membered bicyclic heterocycloalkyl” refers to a bicyclic heterocycloalkyl group having from 7 to 11 ring atoms. Unless otherwise indicated, a heterocycloalkyl group is unsubstituted.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O— alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

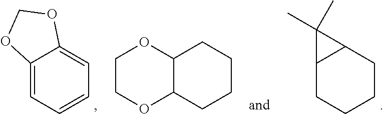

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., alkyl, R¹, R⁷, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Substituted Naphthyridinedione Derivative or a pharmaceutically acceptable salt of the compound. The transformation may occur by various mechanisms (e.g., by metabolic or chemical processes), such as, for example, through hydrolysis in blood. For example, if a Substituted Naphthyridinedione Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Substituted Naphthyridinedione Derivative contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate).

If a Substituted Naphthyridinedione Derivative incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters, including those corresponding to both natural and non-natural amino acids (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Substituted Naphthyridinedione Derivatives can form salts which are also within the scope of this invention. Reference to a Substituted Naphthyridinedione Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Substituted Naphthyridinedione Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Substituted Naphthyridinedione Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Substituted Naphthyridinedione Derivatives may be atropisomers (e.g., substituted biaryls) and are considered as part of this invention. Enantiomers can also be directly separated using chiral chromatographic techniques.

It is also possible that the Substituted Naphthyridinedione Derivatives may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Substituted Naphthyridinedione Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Substituted Naphthyridinedione Derivatives, and of the salts, solvates, hydrates, esters and prodrugs of the Substituted Naphthyridinedione Derivatives, are intended to be included in the present invention.

### GENERAL LIST OF ABBREVIATIONS

ACN=acetonitrile
AcOH=acetic acid
Alk=alkyl
Ar=aryl
Boc=tert-butoxycarbonyl
BOP=benzotriazol-1-yloxytris(dimethylamino)-phosphonium hexafluorophosphate
br=broad
d=doublet
DABCO=1,4-diazabicyclo(2,2,2)octane
DBU=1,8-diazabicyclo[5.4.0]undec-7-ene
DCE=1,2-dichloroethane
DEA=N,N-diethylamine
DIPEA=N,N-diisopropylethylamine
DMA=N,N-dimethylacetamide
DMF=dimethylformamide
DMSO=dimethyl sulfoxide
EDCI=1-ethyl-3-(3′-dimethylaminopropyl)carbodiimide hydrochloride
ESI=electrospray ionization
EtOAc=ethyl acetate
EtOH=ethanol
HATU=2-(1H-7-Azabenzotriazol-1-yl)-1,1,3,3-tetramethyl uronium hexafluorophosphate
HOAc=acetic acid
HOAt=1-hydroxy-7-azabenzotriazole
HOBt=1-hydroxybenzotriazole
HRMS=high resolution mass spectrometry
IPAc=iso-propyl acetate
LCMS=liquid chromatography/mass sepectrometry
M=multiplet
mCPBA=meta-chloroperoxybenzoic acid
MeCN=acetonitrile
MeOH=methyl alcohol
MeTHF=2-methyltetrahydrofuran
min=minutes
MS=mass spectroscopy
MTBE=methyl tert-butyl ether
NMP=1-methyl 2-pyrrolidinone
NMR=nuclear magnetic resonance spectroscopy
PG=protecting group
Piv=pivalate, 2,2-dimethylpropanoyl
Ph=phenyl
rt=room temperature
s=singlet
SFC=supercritical fluid chromatography
T=triplet
TEA=triethylamine
TFA=trifluoroacetic acid
THF=tetrahydrofuran
wt %=weight percent

### The Compounds of Formula (I)

The present invention provides Substituted Naphthyridinedione Derivatives of Formula (I):

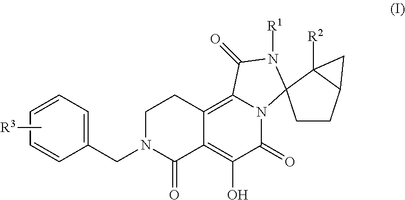

and pharmaceutically acceptable salts thereof, wherein R¹, R² and R³ are defined above for the Compounds of Formula (I).

In one embodiment, for the Compounds of Formula (I), each occurrence of R³ is halo.

In another embodiment, for the Compounds of Formula (I), R³ represents two halo substituents.

In another embodiment, for the Compounds of Formula (I), R³ represents two halo substituents, one of which is F and the other of which is Cl.

In one embodiment, the Compounds of formula (I) have the formula (Ia):

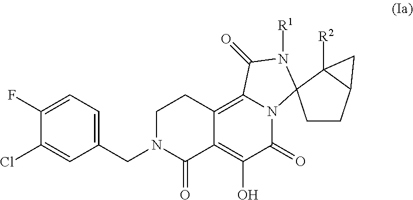

and pharmaceutically acceptable salts thereof,
wherein:

R¹ is H or C₁-C₄ alkyl, wherein said C₁-C₄ alkyl group can be optionally substituted with up to two groups, each independently selected from —OH, F, —OP(O)(OH)₂ and —OC(O)(C₁-C₆ alkyl), wherein the C₁-C₆ alkyl moiety of said —OC(O)(C₁-C₆ alkyl) substituent group can be optionally substituted with up to 2 groups, each independently selected from —NH₂, —N(CH₃)₂, —C(O)NH₂ and —SCH₃;

R² is H or —CH₂R⁴; and

R⁴ is selected from —OH, —SCH₃, —SO₂CH₃ and —OP(O)(OH)₂.

In one embodiment, for the Compounds of Formula (I) or (Ia), R¹ is H.

In another embodiment, for the Compounds of Formula (I) or (Ia), R¹ is C₁-C₄ alkyl, which can be optionally substituted as set forth above for the Compounds of Formula (I).

In another embodiment, for the Compounds of Formula (I) or (Ia), R¹ is selected from H, methyl, —CH₂CH(OH)CH₃, —CH₂CH₂CH(OH)CH₃, —CH₂CH(OH)CH₂F, —CH₂CH(F)CH₂OH, —CH₂CH(—OP(O)(OH)₂)CH₃, —OC(O)CH(NH₂)CH₂CH(CH₃)₂, —OC(O)CH(CH₃)—NH₂, —OC(O)CH(NH₂)CH₂CH₂C(O)NH₂, —OC(O)CH(NH₂)CH₂CH₂SCH₃, —OC(O)CH(isopropyl)-NH₂ and —OC(O)CH₂N(CH₃)₂.

In another embodiment, for the Compounds of Formula (I) or (Ia), R¹ is methyl.

In one embodiment, for the Compounds of Formula (I) or (Ia), R² is H.

In another embodiment, for the Compounds of Formula (I) or (Ia), R² is —(C₁-C₃ alkylene)-R⁴.

In another embodiment, for the Compounds of Formula (I) or (Ia), R² is selected from H, —CH₂OH, —CH₂SCH₃, —CH₂SO₂CH₃ and —CH₂OP(O)(OH)₂.

In one embodiment, for the Compounds of Formula (I) or (Ia), R⁴ is —OH.

In another embodiment, for the Compounds of Formula (I) or (Ia), R⁴ is —OP(O)(OH)₂.

In another embodiment, for the Compounds of Formula (I) or (Ia), R¹ is selected from H, methyl, —CH₂CH(OH)CH₃, —CH₂CH₂CH(OH)CH₃, —CH₂CH(OH)CH₂F, —CH₂CH(F)CH₂OH, —CH₂CH(—OP(O)(OH)₂)CH₃, —OC(O)CH(NH₂)CH₂CH(CH₃)₂, —OC(O)CH(CH₃)—NH₂, —OC(O)CH(NH₂)CH₂CH₂C(O)NH₂, —OC(O)CH(NH₂)CH₂CH₂SCH₃, —OC(O)CH(isopropyl)-NH₂ and —OC(O)CH₂N(CH₃)₂, and), R² is selected from H, —CH₂OH, —CH₂SCH₃, —CH₂SO₂CH₃ and —CH₂OP(O)(OH)₂.

In still another embodiment, for the Compounds of Formula (I) or (Ia), R¹ is methyl or —CH₂CH(—OP(O)(OH)₂)CH₃ and R² is H or CH₂OH.

In one embodiment, variables R¹, R² and R³ for the Compounds of Formula (I) are selected independently of each other.

In another embodiment, the Compounds of Formula (I) are in substantially purified form.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of HIV antiviral agents, immunomodulators, and anti-infective agents.

(c) The pharmaceutical composition of (b), wherein the HIV antiviral agent is an antiviral selected from the group consisting of HIV protease inhibitors, HIV integrase inhibitors, nucleoside reverse transcriptase inhibitors, CCR5 co-receptor antagonists and non-nucleoside reverse-transcriptase inhibitors.

(d) A pharmaceutical combination that is (i) a Compound of Formula (I) and (ii) a second therapeutic agent selected from the group consisting of HIV antiviral agents, immunomodulators, and anti-infective agents; wherein the Compound of Formula (I) and the second therapeutic agent are each employed in an amount that renders the combination effective for inhibiting HIV replication, or for treating HIV infection and/or reducing the likelihood or severity of symptoms of HIV infection.

(e) The combination of (d), wherein the HIV antiviral agent is an antiviral selected from the group consisting of HIV protease inhibitors, HIV integrase inhibitors, nucleoside reverse transcriptase inhibitors, CCR5 co-receptor antagonists and non-nucleoside reverse-transcriptase inhibitors.

(f) A method of inhibiting HIV replication in a subject in need thereof which comprises administering to the subject an effective amount of a Compound of Formula (I).

(g) A method of treating HIV infection and/or reducing the likelihood or severity of symptoms of HIV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HIV antiviral agents, immunomodulators, and anti-infective agents.

(i) The method of (h), wherein the HIV antiviral agent is an antiviral selected from the group consisting of HIV protease inhibitors, HIV integrase inhibitors, nucleoside reverse transcriptase inhibitors, CCR5 co-receptor antagonists and non-nucleoside reverse-transcriptase inhibitors.

(j) A method of inhibiting HIV replication in a subject in need thereof which comprises administering to the subject the pharmaceutical composition of (a), (b) or (c) or the combination of (d) or (e).

(k) A method of treating HIV infection and/or reducing the likelihood or severity of symptoms of HIV infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine, (b) inhibiting HIV replication or (c) treating HIV infection and/or reducing the likelihood or severity of symptoms of HIV infection. In these uses, the compounds of the present invention can optionally be employed in combination with one or more second therapeutic agents selected from HIV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

The Compounds of Formula (I) may be referred to herein by chemical structure and/or by chemical name. In the instance that both the structure and the name of a Compound of Formula (I) are provided and a discrepancy is found to exist between the chemical structure and the corresponding chemical name, it is understood that the chemical structure will predominate.

Non-limiting examples of the Compounds of Formula (I) include compounds 1-19 as set forth below, and pharmaceutically acceptable salts thereof

The compounds of the present invention can be readily prepared according to the following reaction schemes and examples, or modifications thereof, using readily available starting materials, reagents and conventional synthetic procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes 1-6 below. Alternative synthetic pathways and analogous structures will be apparent to those skilled in the art of organic synthesis.

Scheme 1 depicts a method for preparing compounds of the present invention, wherein benzyl halide A is reacted with amide B in the presence of a base. The corresponding tertiary amide is deprotonated and reacted with phenyl methyl sulfone to generate sulfoxide C. The sulfoxide is then converted to the corresponding α,β-unsaturated product via a Pummerer rearrangement and sulfide oxidation to compound D. Reaction with the anion of a protected amino ester and acid hydrolysis affords the alpha amino ester E. Elimination of the sulfone under basic conditions affords the unsaturated ester F. Conversion of F to the pyridinone compound G can be accomplished in the presence of an ester oxalyl chloride followed by treatment with DABCO and LiBr. Hydrolysis of ester G to the acid affords compound H which is then transformed to the tris-pivalate J using standard conditions. Compounds H and J serve as valuable intermediates which can be transformed to HIV integrase inhibitors using the procedures and methods described in the following schemes.

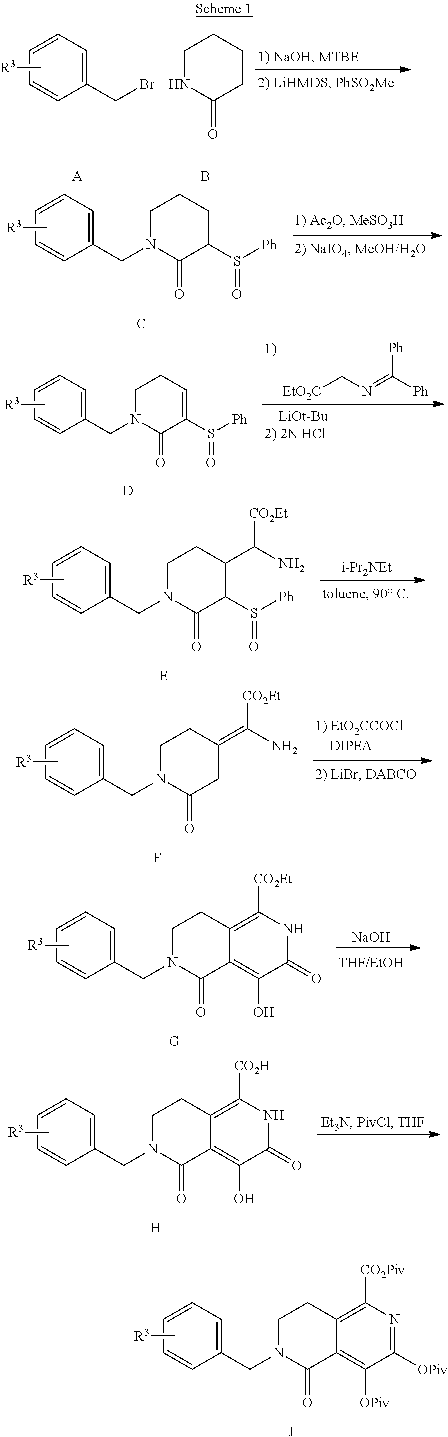

Scheme 2 depicts a method for preparing compounds in the present invention, wherein activated mixed anhydride J is reacted with primary amine K to yield secondary amide compound L. Conversion of L to the desired product O can be conducted by acid-mediated condensation onto an aldehyde or ketone (M) or ketal (N).

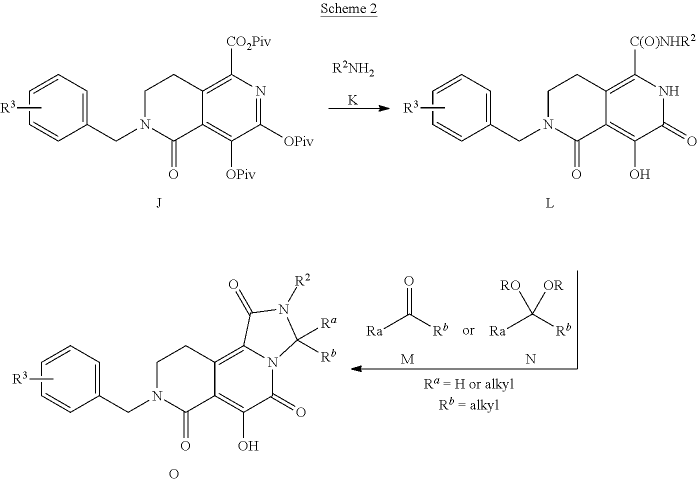

Scheme 3 depicts a general method for preparing phosphate prodrugs of compounds of the general structure O bearing a primary or secondary hydroxyl group on the R⁴ substituent. Treatment of O with diphosphoryl chloride, followed by base-mediated hydrolysis and acidic work-up furnishes clean conversion to the desired phosphaste prodrug (P), which is stable enough to be chromatographed under reverse phase conditions.

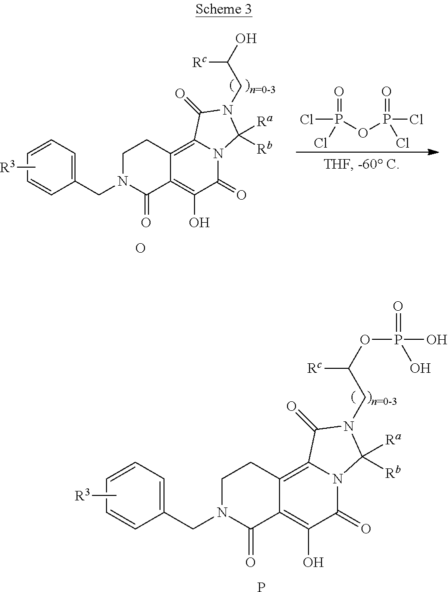

Scheme 4 depicts a method for preparing aminoester prodrugs of compounds of the general structure O bearing a primary or secondary hydroxyl group on the R⁴ substituent. Treatment of O with N-Boc protected amino acids under appropriate coupling conditions furnished protected intermediate Q, which can be readily deprotected under acidic conditions to afford the corresponding HX salt of prodrug R.

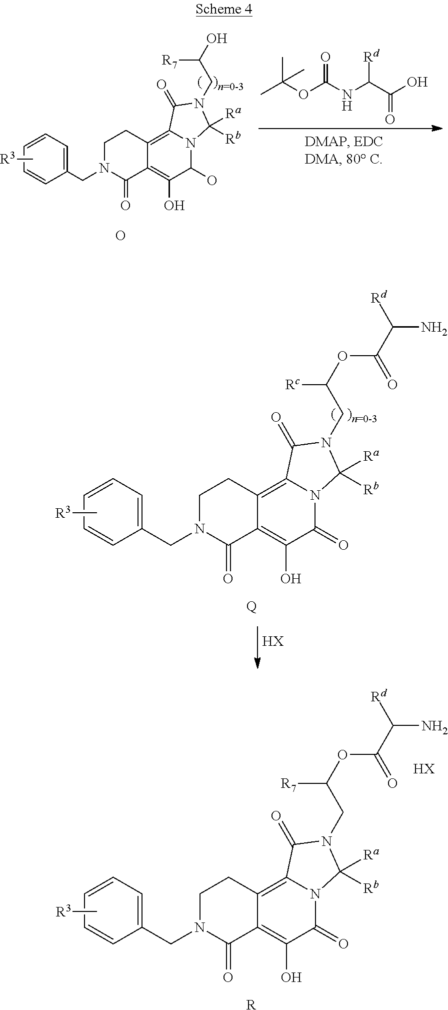

Scheme 5 depicts a method for preparing compounds in the present invention, wherein activated mixed anhydride J is reacted with ammonium hydroxide to yield primary amide compound S. Conversion of S to the desired product V can be conducted by acid-mediated condensation onto an aldehyde or ketone (T) or ketal (U).

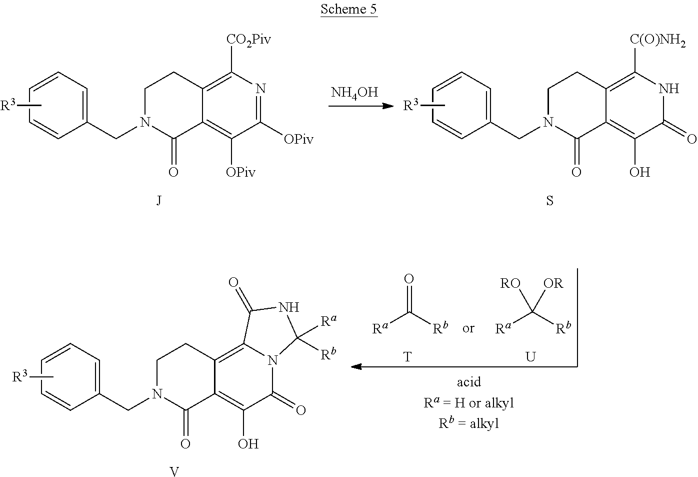

Scheme 6 depicts a method for preparing compounds in the present invention wherein intermediate V is reacted with an alkyl halide W to form the desired N-alkylated amide X.

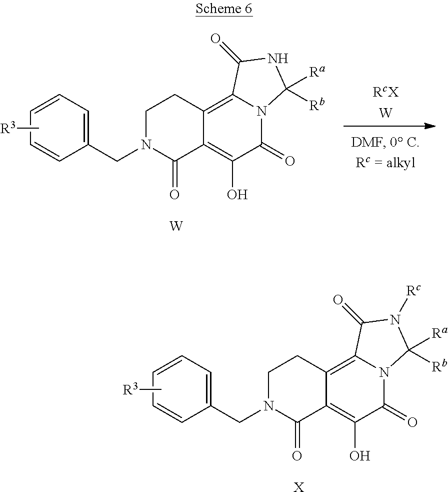

In the methods for preparing compounds of the present invention set forth in the foregoing schemes, functional groups in various moieties and substituents (in addition to those already explicitly noted in the foregoing schemes) may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. The protecting groups may be removed at a convenient subsequent stage using methods known in the art. Alternatively the interfering group can be introduced into the molecule subsequent to the reaction Step of concern.

One skilled in the art of organic synthesis will recognize that the synthesis of compounds with multiple reactive functional groups, such as —OH and NH₂, may require protection of certain functional groups (i.e., derivatization for the purpose of chemical compatibility with a particular reaction condition). Suitable protecting groups for the various functional groups of these compounds and methods for their installation and removal are well-known in the art of organic chemistry. A summary of many of these methods can be found in Greene & Wuts, Protecting Groups in Organic Synthesis, John Wiley & Sons, 3^{rd} edition (1999).

One skilled in the art of organic synthesis will also recognize that one route for the synthesis of the Compounds of Formula (I) may be more desirable depending on the choice of appendage substituents. Additionally, one skilled in the relevant art will recognize that in some cases the order of reactions may differ from that presented herein to avoid functional group incompatibilities and thus adjust the synthetic route accordingly.

Compounds of formula O, P, Q, R, V and W may be further elaborated using methods that would be well-known to those skilled in the art of organic synthesis or, for example, the methods described in the Examples below, to make the full scope of the Compounds of Formula (I).

The starting materials used and the intermediates prepared using the methods set forth in Schemes 1-6 may be isolated and purified if desired using conventional techniques, including but not limited to filtration, distillation, crystallization, chromatography and alike. Such materials can be characterized using conventional means, including physical constants and spectral data.

### EXAMPLES

### General Methods

The following examples serve only to illustrate the invention and its practice. The examples are not to be construed as limitations on the scope or spirit of the invention. In these examples, all temperatures are degrees Celsius unless otherwise noted, and “room temperature” refers to a temperature in a range of from about 20° C. to about 25° C. Mass spectra (MS) were measured by electrospray ion-mass spectroscopy (ESI). ¹H NMR spectra were recorded on Varian or Bruker instruments at 400-500 MHz. Compounds described herein were synthesized as a racemic mixture unless otherwise stated in the experimental procedures.

### Example 1

### Preparation of Intermediate Compound Int-1g

### Step A—Synthesis of Intermediate Compound Int-1a

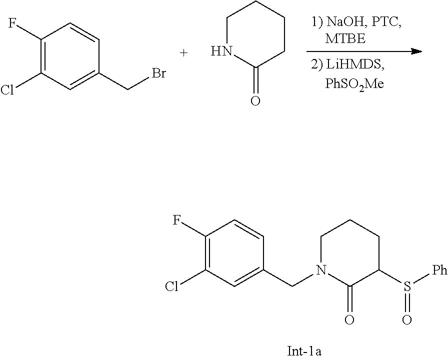 [SMILES: Fc1ccc(CBr)cc1Cl ; O=C1CCCCN1 ; O=C1C(S(=O)c2ccccc2)CCCN1Cc1ccc(F)c(Cl)c1]

To a 2000 L glass-lined reactor, under the protection of nitrogen, MTBE (633 kg) and valerolactam (34.9 kg, 350 mol) were charged by vacuum. After initiating stirring, a 33% aqueous solution of tetrabutylammonium hydrogen sulfate (8.35 kg) was added. The mixture was cooled to 20-30° C. and then a 50% aqueous solution of sodium hydroxide (270 kg) was added to the mixture at a rate of 10-15 L/minute at this temperature. After the addition, the mixture was maintained at the same temperature for 30 minutes followed by the addition of 3-chloro-4-fluoro-benzylbromide (62.9 kg, 280 mol) at a rate of 2-3 kg/minute at 20-30° C. After 5-10 hours, water (283 kg) was added to the reaction mixture at a rate of 30-40 kg/minute at 20-30° C. to quench the reaction. The mixture was stirred for 30 minutes and then the water phase was separated out. The organic phase was washed with 25% aqueous brine solution (226 kg), and the organic phase was dried with anhydrous sodium sulfate (30 kg) under stirring. The dried mixture was filtered by nutsche filter and the filter cake was rinsed with MTBE (50 kg). The combined filtrate was concentrated in vacuo (T≦35° C., P≦−0.08 MPa) until the mixture volume remained at about 350-500 L. Petroleum ether was added (138.4 kg) to the mixture and concentrated continuously. After the mixture volume remained at about 350-500 L, another 138 Kg of petroleum ether was added to the mixture and then concentrated in vacuo. The mixture was cooled to 0-5° C., stirred for 2-3 hours, and then filtered. The filter cake was dried by rotary conical dryer below 35° C. to provide 67.7 kg of 1-(3-chloro-4-fluorobenzyl)piperidin-2-one (99% yield).

To a 1500 L low temperature reactor, THF (117.5 kg) and hexamethyl disilylamine (73.5 kg, 455 mol) were charged. The mixture was cooled down to −30 to −20° C. To the solution was added n-BuLi (130.6 kg, 455 mol) at a rate of 50-60 kg/hour at −30 to −20° C. After the addition, the reaction mixture was maintained at the same temperature for 30 minutes. Under the protection of nitrogen, to another 1500 L low temperature reactor, THF (222.5 kg) was charged, followed by the benzyl lactam from the above Step (50 kg, 207 mol). The mixture was cooled to −30 to −20° C. and then the THF solution of LiHMDS which was prepared in advance was added at the rate of 100-150 L/h at −30 to −20° C. The reaction mixture was maintained at this temperature for 6 hours until the reaction was complete as monitored by HPLC analysis. Methyl phenyl sulfone (45.7 kg, 228 mmol) was added into the reaction mixture at a rate of 5-10 kg/hour at −30 to −20° C. After the addition, the reaction mixture was maintained at this temperature for 1 hour until HPLC analysis revealed complete consumption of the sulfone starting material. Under the protection of nitrogen, 4 N aqueous HCl solution was added to quench the reaction at a temperature of −5 to 5° C. Ethyl acetate (424 kg) was added into the mixture and the water phase was separated out (repeated twice). The combined organic phases were washed twice with water (170 kg) and 25% brine (2×204 kg), dried for 8 hours with anhydrous sodium sulfate (50 kg) and filtered by nutsche filter. The filter cake was rinsed with ethyl acetate (50 kg) for 30 minutes, and then combined with the filtrate. The filtrate was concentrated in vacuo (T≦30° C., P≦−0.08 MPa) until a volume of about 300-350 L of the mixture remained. MTBE (340 kg) was added into the concentrated liquor and then concentration was continued until 150-200 L volume of the mixture remained. Petroleum ether (73 kg) was added into the concentrated liquors under stirring, and then the mixture was cooled to 0° C. to induce crystallization. The crystallized mixture was filtered by nutsche filter under the protection of nitrogen. The filter cake was rinsed with the mixed solvent of MTBE (20 kg) and petroleum ether (24 kg) to provide the compound Int-1a as a white solid.

### Step B—Synthesis of Intermediate Compound Int-1b

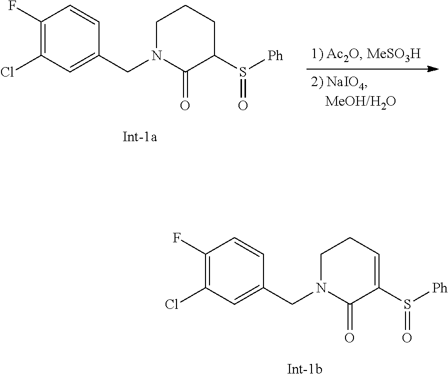 [SMILES: O=C1C(S(=O)c2ccccc2)CCCN1Cc1ccc(F)c(Cl)c1 ; O=C1C(S(=O)c2ccccc2)=CCCN1Cc1ccc(F)c(Cl)c1]

Under the protection of nitrogen, to a clean and dry 1000 L glass-lined reactor, was charged acetonitrile (340 kg), followed by Int-1a (70 kg, 190 mol). Under stirring, acetic anhydride (39.1 kg, 380 mol) was added to the mixture at the rate of 10 kg/minute, and then methanesulfonic acid (9.2 kg, 100 mol) was added at the rate of 1 kg/minute at 18 to 28° C. After the addition, the mixture was stirred at this temperature for 10-15 hours until complete as determined by HPLC analysis. The mixture was then concentrated in vacuo (T≦30° C., P≦−0.08 MPa). Under the protection of nitrogen, deoxygenated ethyl acetate (315 kg) was added, the mixture was washed with 5% brine (3×315 kg) and then water phase was separated out. The organic phase was concentrated in vacuo (T≦30° C., P≦−0.08 MPa) after which deoxygenated methanol (486 kg), deoxygenated purified water (124.6 kg) and sodium periodate (102.2 kg, 480 mol) were added to the mixture and the mixture stirred at 18 to 28° C. for 26 hours. The mixture was filtered by nutsche filtration and the filter cake was rinsed with dichloromethane (2×133 kg), and then the filtrates were combined. The filtrate was diluted with water (700 kg) and then extracted with dichloromethane (2×455 kg). The combined organic layers were washed with 15% brine (378 kg), dried with anhydrous sodium sulfate (21 kg) and filtered by nutsche filtration. The filter cake was rinsed with dichloromethane (2×35 kg) and the mother liquors were concentrated in vacuo (T≦30° C., P≦−0.08 MPa) until the remaining mixture volume was about 200-250 L. Isopropyl alcohol (138 kg) was added and the mixture was concentrated continuously. After the remaining mixture volume was about 200-250 L, a mixture of isopropyl alcohol (35 kg) and petroleum ether (30 kg) was added, and then the mixture was concentrated for the third time. After the concentration was completed, petroleum ether (168 kg) was added. Then mixture was cooled to −5 to 0° C. to induce crystallization. The mixture was filtered by centrifuge and the filter cake was dried to provide compound Int-1b (59.4 kg, 85% yield).

### Step C—Synthesis of Intermediate Compound Int-1c

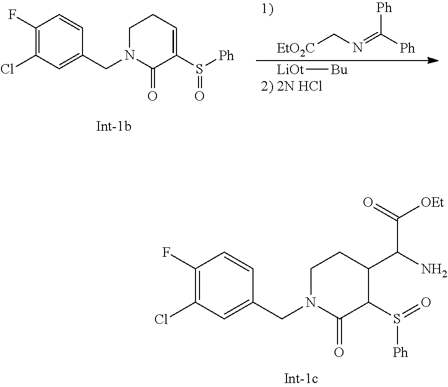

To a 100 L flask was added THF (40 L), followed by Int-1b (5.0 kg, 13.7 mol) and the commercially available ethyl 2-(diphenylmethyleneamino)acetate (4.0 kg, 15.1 mol). The batch was stirred at room temperature to dissolve the solids and then cooled to 0° C. in an ice/water bath. Lithium tert-butoxide (1.4 L, 1 M in THF) was then added dropwise, maintaining the temperature below 15° C. The batch was stirred at 0° C. for 1 hour until full conversion is evidenced by HPLC analysis. To the cooled batch was added 2M aqueous HCl solution (35 L) at a rate that allows the batch to warm gradually to room temperature (15 minutes). The hazy yellow solution was then stirred at room temperature for 30-45 minutes. The solution was charged to a 200 L extractor, and MTBE (25 L) was added. Layers were separated, and the organic layer was extracted with 2 M aqueous HCl solution (5 L). The combined aqueous layers were washed with MTBE (2×25 L) to remove residual benzophenone. The acidic aqueous layer was recharged to a 100 L flask, along with Isopropyl acetate (25 L) and the batch was cooled to 0° C. Aqueous 5M NaOH solution (˜25 L) was added dropwise, keeping the temperature below 5° C., until the pH was 8.5. Layers were then separated, and the aqueous layer was re-extracted with Isopropyl acetate (8 L) and the resulting Isopropyl acetate solution, which contains compound Int-1c, was used as is in the next step.

### Step D—Synthesis of Intermediate Compound Int-1d

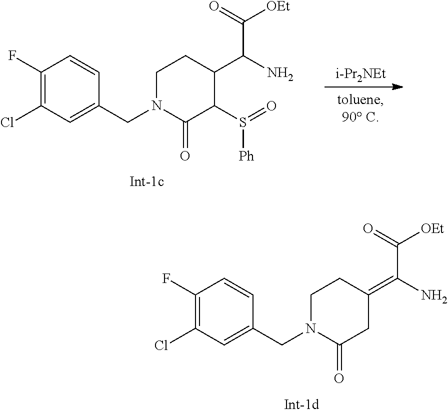 [SMILES: "i-Pr2NEt" ; CCOC(=O)C(N)C1CCN(Cc2ccc(F)c(Cl)c2)C(=O)C1S(=O)c1ccccc1 ; CCOC(=O)/C(N)=C1\CCN(Cc2ccc(F)c(Cl)c2)C(=O)C1]

To a 100 L flask was added compound Int-1c (6.4 kg, 13.7 mol) as a solution in Isopropyl acetate. The batch was solvent switched to toluene then adjusted to a total volume of 65 L (KF=200 ppm). Hunig's base (2.4 L, 13.8 mol) was added, along with a water-cooled condenser and the slurry was heated to 90° C. After 30 minutes at 90° C., the batch was assayed for conversion and then cooled slightly. Batch concentration commenced at ˜70° C. and the volume was reduced to 18 L, upon which a slurry formed. Once the appropriate volume is reached, Isopropyl acetate (2 L) was added in a single addition, and the slurry was slowly cooled to room temperature, and stirred until the supernatant concentration was below 16 mg/mL. The slurry was filtered, rinsed with 5:1 heptane:Isopropyl acetate (12 L), and dried overnight on the filter pot with vacuum and nitrogen sweep to provide compound Int-1d as a fluffy white solid (3.0 Kg, 65% isolated yield).

### Step E—Synthesis of Intermediate Compound Int-1e

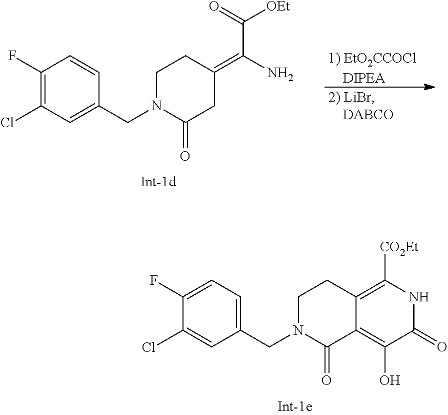

In a 100 L flask was charged with compound Int-1d (3.50 kg, 8.80 mol) and THF (45 L). The batch was cooled to 0° C. and DIPEA was added (1.70 L, 1.4 mol). To the resulting solution was added dropwise the monoethyl oxalyl chloride (1.2 L, 9.24 mol) at such a rate that the temperature is maintained below 3.5° C. (45 minutes to 1 h). After stirring the reaction mixture for 30 minutes below 3.5° C., the reaction was monitored for completion by HPLC analysis. To the batch was added directly, as a solid, LiBr (3.06 kg, 35.2 mol) followed by DABCO (1.97 kg, 17.6 mol). The batch was allowed to warm to room temperature and stirred overnight (16 h) at room temperature. The reaction mixture was quenched with 2 M aqueous HCl solution (35 L) and stirred at room temperature for 30 minutes. Approximately half to three quarters of the total THF was then removed in vacuo, and the resulting slurry was diluted to the original quench volume with water. The approximate amount of THF removed was 36-38 L. The slurry was stirred at room temperature for 30 minutes and filtered. The wet cake was washed with water (2×12 L) and then with MTBE (3×12 L) and dried under vacuum/N₂ sweep until dry, affording compound Int-1e.

### Step F—Synthesis of Intermediate Compound Int-1f

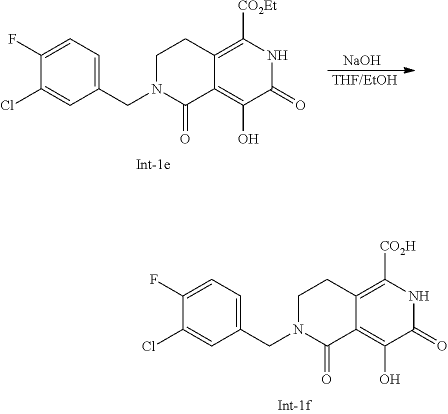

A 100 L flask was charged with compound Int-1e (3.08 kg, 7.80 mol) and 37 L of a 1:1 mixture of EtOH/THF. To the resulting slurry was added 9.4 L of a 5 M aqueous NaOH solution and the batch was warmed to 50-53° C. for 45 minutes. The slurry was then diluted with 10 L of water (˜3.33 L/Kg) and stirred for an additional 1 hour at 50-53° C. Upon completion of the hydrolysis, the batch was cooled to 15° C. and acidified with 6 L of concentrated HCl and stirred at room temperature for 12 hours until the reaction was complete as monitored by HPLC analysis. The slurry was then filtered, washed with water (3×12 L) and dried under vacuum/N₂ sweep at 35° C. until dry to provide compound Int-1f as a colorless solid.

### Step G—Synthesis of Intermediate Compound Int-1g

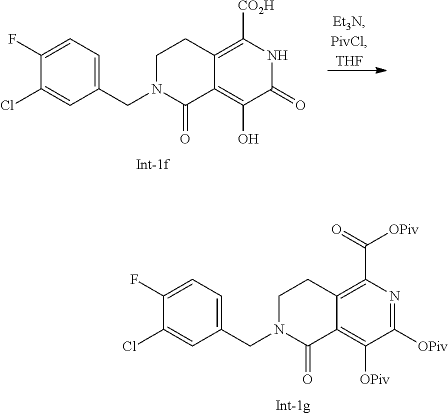

A 100 mL flask was charged with 15 mL of THF (KF<300 ppm) and compound Int-1f (2.73 mmol, 1.0 g). Triethylamine (1.90 mL, 13.65 mmol) was then added under nitrogen at 20° C. The slurry was cooled to 10° C. and trimethylacetyl chloride (1.18 mL, 9.56 mmol) added. The slurry was then stirred at 20° C. for 5 hours until HPLC analysis revealed complete conversion. The slurry was filtered (to remove Et₃N/HCl salt) and the solid washed with 5 mL of dry THF. The solid was discarded and the filtrate then solvent switch in vacuo to heptane with a final volume of 15 mL. The resultant slurry was stirred at 20° C. for 1 hour, filtered, washed with one bed of heptane and dried in oven at 40° C. with a nitrogen stream for 12 hours to provide compound Int-1g. MS (+ESI) m/z=619.

### Example 2

### Preparation of Intermediate Compound Int-2e

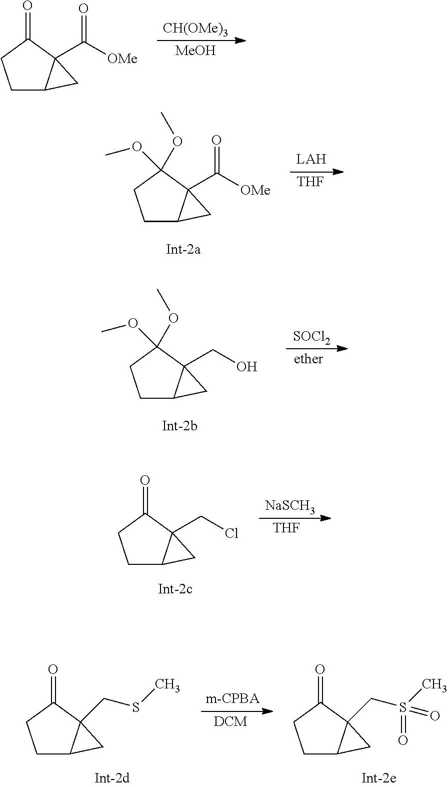

### Step A—Synthesis of Intermediate Compound Int-2a

To a 20° C. solution of methyl 2-oxobicyclo[3.1.0]hexane-1-carboxylate (3.08 g, 20.0 mmol, made racemically using the method described in Synlett 2007, 4, 579-582) in MeOH (20 mL) was added trimethyl orthoformate (2.5 mL, 22.0 mmol) and cerium (III) trifluoromethanesulfonate (120 mg, 0.20 mmol). The reaction was allowed to stir at 20° C.-30° C. for 2 hours, then was quenched with TEA (0.5 mL). The resulting solution was diluted with MTBE (150 mL), washed with brine (50 mL), dried over Na₂SO₄, filtered and concentrated in vacuo to provide compound Int-2a (3.1 g, yield: 77%) as pale yellow oil. (300 MHz, CDCl₃) δ 3.64 (s, 3H), 3.45 (s, 3H), 3.29 (s, 3H), 2.03-2.10 (m, 2H), 1.87-1.97 (m, 1H), 1.66-1.71 (m, 1H), 1.27-1.37 (m, 2H), 1.09 (t, J=5.4 Hz, 1H).

### Step B—Synthesis of Intermediate Compound Int-2b

To a mixture of LiAlH₄ (2.05 g, 0.054 mol) in THF (210 mL) at −5° C. was added a solution of compound Int-2b (7.2 g, 0.036 mol) in THF (80 mL) dropwise. After addition, the mixture was stirred at −5° C.-0° C. for 2 hours. The mixture was quenched with water (2.05 mL), 15% NaOH (2.05 mL) and water (6.15 mL) carefully. The mixture was filtered and the cake washed with EtOAc (250 mL), dried over Na₂SO₄, concentrated in vacuo to provide compound Int-2b (6.1 g, yield: 98%) as pale yellow oil. (300 MHz, CDCl₃) δ 4.39 (d, J=11.7 Hz, 1H), 3.41 (s, 3H), 3.29 (s, 3H), 3.18-3.29 (m, 1H), 3.04-3.11 (m, 1H), 1.89-2.08 (m, 2H), 1.65-1.72 (m, 1H), 1.44-1.51 (m, 1H), 1.31-1.38 (m, 1H), 0.72 (t, J=5.1 Hz, 1H), 0.61-0.66 (m, 1H).

### Step C—Synthesis of Intermediate Compound Int-2c

To a solution of compound Int-2b (5.1 g, 0.030 mmol) in anhydrous ether (100 mL) was added SOCl₂ (3.9 g, 0.033 mmol) by syringe at 0° C. under N₂ atmosphere and stirred overnight. The mixture was quenched with water (50 mL) at 0° C. and extracted with ether (150 mL). The organic layer was dried over Na₂SO₄ and concentrated in vacuo to provide compound Int-2c (6.0 g) which was used directly for next step without further purification. (300 MHz, CDCl₃) δ 4.01 (d, J=15.6 Hz, 1H), 3.43 (d, J=15.6 Hz, 1H), 2.11-2.22 (m, 1H), 2.02-2.08 (m, 3H), 1.90-1.99 (m, 1H), 1.28-1.36 (m, 1H), 1.18 (t, J=5.1 Hz, 1H).

### Step D—Synthesis of Intermediate Compound Int-2d

To a crude solution of compound Int-2c (6.0 g) in THF (100 mL) at 0° C. was added NaSCH₃ (6.3 g, 0.090 mmol) under N₂ atmosphere and stirred overnight. The mixture was quenched with water (20 mL) at 0° C. and extracted with DCM (3*50 mL). The organic layer was dried over Na₂SO₄ and concentrated in vacuo to provide a residue, which was purified using silica gel column (petroleum ether:ethylacetate=30:1 to 20:1) to provide compound Int-2d (2.1 g, yield: 45% for two steps) as a yellow oil. (300 MHz, CDCl₃) δ 3.07 (d, J=13.8 Hz, 1H), 2.68 (d, J=13.8 Hz, 1H), 2.13-2.20 (m, 7H), 1.96-1.99 (m, 1H), 1.30-1.34 (m, 1H), 1.14 (t, J=4.5 Hz, 1H).

### Step E—Synthesis of Intermediate Compound Int-2e

To a solution of compound Int-2d (1.2 g, 7.68 mmol) in DCM (15 mL) was added m-CPBA (3.12 g, 15.36 mmol) at 0° C. The mixture was stirred at room temperature overnight, then the solvent was removed in vacuo and the residue obtained was purified using silica gel column (petroleum ether:ethylacetate=5:1) to provide compound Int-2e (0.80 g, yield: 55%) as yellow oil. ¹H NMR (400 MHz, CDCl₃) δ 3.55 (d, J=14.8 Hz, 1H), 3.23 (d, J=15.2 Hz, 1H), 2.96 (s, 3H), 2.47-2.52 (m, 1H), 2.17-2.25 (m, 4H), 1.61-1.65 (m, 1H), 1.34 (t, J=4.2 Hz, 1H).

### Example 3

### Preparation of Intermediate Compound Int-3c

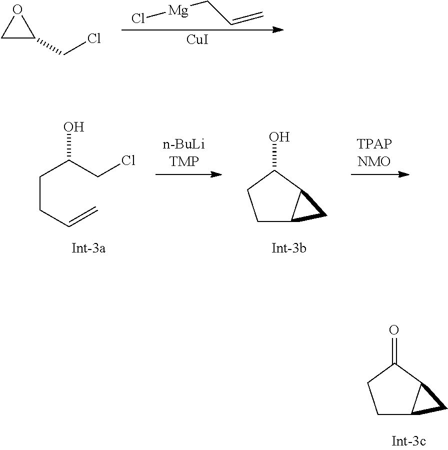

### Step A—Synthesis of Intermediate Compound Int-3a

To an oven-dried flask under an atmosphere of nitrogen was added (S)-2-(chloromethyl)oxirane (25.0 g, 270 mmol), anhydrous THF (270 mL) and copper (I) iodide (5.15 g, 27.0 mmol). The mixture was cooled to −78° C. in a dry ice/acetone bath. A solution of allyl magnesium chloride (2.0 M in THF, 149 mL, 298 mmol) was added over 25 minutes to the reaction. The reaction was slowly warmed to −10° C. over 2 hours and the cooling bath was then removed. The reaction was stirred further for 3 hours at room temperature. Aqueous NH₄Cl was added to quench the reaction, the reaction was poured into a separatory funnel and extracted with diethyl ether (3×200 mL). The combined organic layers were dried over Na₂SO₄, filtered through a plug of silica gel, and concentrated in vacuo to provide compound Int-3a (33 g, 91% yield). ¹H NMR (400 MHz, CDCl₃) δ 5.82 (ddt, J=17.2, 10.2, 6.6 Hz, 1H), 5.07 (dq, J=17.2, 1.7 Hz, 1H), 5.01 (dq, J=10.2, 1.7 Hz, 1H), 3.85-3.80 (m, 1H), 3.64 (dd, J=11.1, 3.5 Hz, 1H), 3.49 (dd, J=11.1, 7.0 Hz, 1H), 2.27-2.15 (m, 3H), 1.67-1.61 (m, 2H).

### Step B—Synthesis of Intermediate Compound Int-3b

To an oven-dried flask under an atmosphere of nitrogen was added compound Int-3a (33.0 g, 245 mmol), anhydrous MTBE (900 mL), and 2,2,6,6-tetramethylpiperidine (107 mL, 635 mmol). The solution was cooled to −78° C. in a dry ice/acetone bath. A solution of n-butyl lithium (1.6 M in hexanes, 556 mL, 889 mmol) was added over 30 minutes to the reaction. The reaction was slowly warmed to room temperature over 16 hours. The reaction was re-cooled to −20° C. and methanol (50 mL) was added portion-wise. The reaction was warmed to room temperature and diluted with MTBE (300 mL). The reaction was poured into a separatory funnel and the organic layer was washed successively with 2 M aqueous HCl solution (2×400 mL) and then water. The combined aqueous layers were back-extracted with MTBE (2×100 mL). The combined organic layers were dried over Na₂SO₄, filtered, and concentrated in vacuo provide compound Int-3b, which was used without further purification. ¹H NMR (400 MHz, CDCl₃) δ 4.23 (d, J=4.8 Hz, 1H), 2.00-1.88 (m, 1H), 1.68 (dd, J=12.5, 8.1, 1H), 1.55 (dd, J=14.5, 8.4, 1H), 1.45-1.28 (m, 3H), 0.48-0.41 (m, 1H), 0.05-0.01 (m, 1H).

### Step C—Synthesis of Intermediate Compound Int-3c

To a solution of compound Int-3b (24.0 g, 245 mmol) in anhydrous CH₂Cl₂ (1500 mL), was added 4-methylmorpholine-N-oxide (45.8 g, 391 mmol) and 50 g of 3 Å molecular sieves. The reaction was placed in a water bath, tetrapropylammonium perruthenate (4.4 g, 12.5 mmol) was added, and the reaction was stirred at room temperature for 18 hours. The reaction was filtered through a pad of Celite, the filtrate was poured into a separatory funnel and washed successively with 2 M aqueous HCl solution (200 mL) and water (100 mL). The combined aqueous layers were back-extracted with CH₂Cl₂ (2×100 mL). The combined organic layers were dried over Na₂SO₄, filtered through a plug of silica gel, rinsing with 25% diethyl ether/CH₂Cl₂, and concentrated in vacuo. The residue obtained was then purified using vacuum distillation to provide a crude residue which was distilled at 60-65° C. at a vacuum of 10 mm Hg to provide compound Int-3c (9.36 g, 44% yield). ¹H NMR (400 MHz, CDCl₃) δ 2.18-1.98 (m, 5H), 1.80-1.74 (m, 1H), 1.23-1.16 (m, 1H), 0.93 (q, J=4.0 Hz, 1H).

### Example 4

### Preparation of Intermediate Compound Int-4a

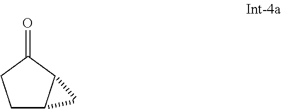

Compound Int-4a was prepared using the method described above in Example 1, Step A and replacing (S)-2-(chloromethyl)oxirane with (R)-2-(chloromethyl)oxirane in Step 1. ¹H NMR (400 MHz, CDCl₃) δ 2.18-1.98 (m, 5H), 1.80-1.74 (m, 1H), 1.23-1.16 (m, 1H), 0.93 (q, J=4.0 Hz, 1H).

### Example 5

### Preparation of Compound 1

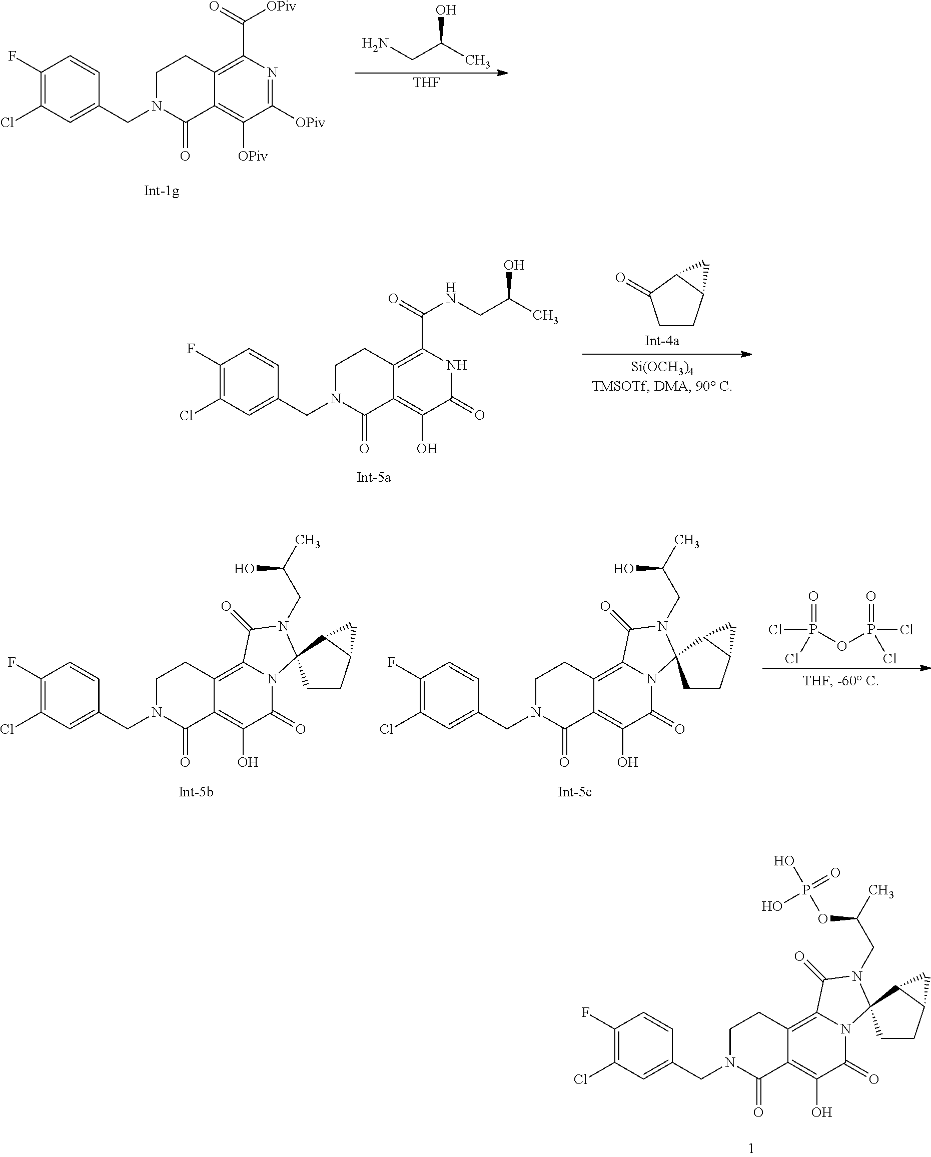

### Step A—Synthesis of Intermediate Compound Int-5a

Compound Int-1g (30 g, 48.5 mmol) was dissolved in THF (190 mL). A THF solution (40 mL) of (S)-(+)-1-amino-2 propanol (14.56 g, 194 mmol) was added over 30 seconds. The reaction was stirred at ambient temperature until the reaction was complete by LCMS analysis. The thick precipitate was collected by filtration, washed with THF (2×250 mL) and dried under vacuum. This solid was then suspended in 1:2 methanol/water (600 mL) and the pH was adjusted to pH=1 with conc. HCl. This mixture was stirred for 2 hours. The resulting white solid was collected by vacuum filtration, and washed with water (250 mL) and compound diethyl ether (2×250 mL). The solid was dried under high vacuum to provide compound Int-5a as a white solid (16.54 g, 77%). LRMS (+ESI) m/z=424.2. ¹H NMR (400 MHz, CD₃OD): δ 7.51 (dd, J=7.0, 2.4 Hz, 1H); 7.37-7.32 (m, 1H); 7.23 (t, J=8.8 Hz, 1H); 4.73 (s, 2H); 3.95-3.86 (m, 1H); 3.52 (t, J=6.5 Hz, 3H); 3.43-3.34 (m, 2H); 3.22 (dd, J=13.8, 7.5 Hz, 1H); 1.19 (d, J=6.3 Hz, 3H).

### Step B—Synthesis of Intermediate Compounds Int-5b and Int-5c

A solution of compound Int-5a (7.5 g, 17.70 mmol) and tetramethoxysilane (5.73 ml, 35.4 mmol) in DMA (44.2 mL) was treated with trimethylsilyl trifluoromethanesulfonate (3.20 ml, 17.70 mmol). The mixture was capped in a pressure vessel and heated to 90° C. for 15 minutes. The mixture was then treated with compound Int-4a (5.10 g, 53.1 mmol), the vessel was sealed, and the reaction was heated to 90° C. and allowed to stir at this temperature for 3 hours. The reaction mixture was cooled to room temperature and was partitioned between water (300 mL) and ethyl acetate (300 mL). A white precipitate formed in organic layer and the organic layer was filtered through paper in a Buchner funnel to remove the solid. The filtered organic phase was washed two more times with water and once with brine. The organic phase was dried over Na₂SO₄, filtered and concentrated in vacuo. Additional solid byproduct was noted to come out of solution upon concentration. The near concentrated slurry was filtered again through paper and the collected solid was washed with EtOAc and DCM and the filtrate was concentrated to provide ˜13 g of an orange oil. The resulting material was purified using gradient elution on reverse phase (50×250 mm (5 um) Sunfire Prep C18; 27-62% CH3CN/water w/0.1% TFA modifier over 30 min @ 90 mL/min). Pure fractions of both products were combined separately and concentrated to remove MeCN until product began to precipitate. The slurries were extracted with dichloromethane (2×150 mL) and the organic phases were dried over Na₂SO₄, filtered, and concentrated in vacuo to provide the isomeric title compounds (Int-5b: 2.2 g, 25% and Int-5c: 1.9 g, 22%) as light yellow foams. Compound Int-5b: LRMS (+ESI) m/z=502.2 found. ¹H NMR (500 MHz, CDCl₃): δ 13.51 (s, 1H); 7.38 (dd, J=6.9, 2.2 Hz, 1H); 7.22 (ddd, J=8.5, 4.5, 2.2 Hz, 1H); 7.15 (t, J=8.6 Hz, 1H); 4.70 (d, J=3.1 Hz, 2H); 4.15 (td, J=6.8, 3.3 Hz, 1H); 3.68 (dd, J=14.8, 3.3 Hz, 1H); 3.56-3.46 (m, 3H); 3.43-3.38 (m, 2H); 2.98-2.90 (m, 1H); 2.14-1.98 (m, 3H); 1.85 (t, J=10.5 Hz, 1H); 1.43-1.38 (m, 1H); 1.29 (d, J=6.3 Hz, 3H); 0.98-0.92 (m, 1H); 0.68-0.64 (m, 1H). Compound Int-5b: LRMS (+ESI) m/z=502.2. ¹H NMR (500 MHz, CDCl₃): δ 13.49 (s, 1H); 7.38 (dd, J=6.9, 2.2 Hz, 1H); 7.23-7.19 (m, 1H); 7.14 (t, J=8.6 Hz, 1H); 4.70 (d, J=5.0 Hz, 2H); 4.23 (ddd, J=8.8, 6.1, 2.7 Hz, 1H); 3.65 (dd, J=14.8, 8.5 Hz, 1H); 3.59-3.38 (m, 6H); 2.18 (d, J=11.9 Hz, 1H); 2.12-1.99 (m, 2H); 1.77-1.68 (m, 2H); 1.29 (d, J=6.3 Hz, 4H); 0.96-0.89 (m, 1H).

### Step C—Synthesis of Compound 1

To compound Int-5b (Diastereomer A, 6.3 g, 12.5 mmol) in dry THF (41.6 mL) cooled to −60° C. was added diphosphoryl choride (3.0 eq, 5.2 mL, 37.4 mmol) dropwise. The mixture stirred at the same temperature for 30 minutes and was then warmed to 0° C. over 10 mins. Water (100 mL) was added followed by a slow addition of a saturated solution of sodium bicarbonate to pH 8. The mixture was then slowly acidified with 1 N HCl to pH 2, and the solid (title compound) was collected by vacuum filtration, washing with water. The solid was dissolved in DMSO and purified using gradient elution on reverse phase (SunFire™ Prep C18 OBD™ 5 um 50×250 mm column; 25-70% CH₃CN/water with 0.1% TFA modifier over 30 minutes) to provide the title compound (5.6 g, 77% yield) as a yellow solid. The solid was redissolved in 1:1 acetontrile/water (2.2 g/600 mL) and slowly evaporated over 4 days under a stream of nitrogen to provide the compound 1 as a crystalline, pale yellow, fluffy solid. LRMS (+ESI) m/z=582.2 found. ¹H NMR (500 MHz, DMSO): δ 7.58 (d, J=7.2 Hz, 1H); 7.42-7.33 (m, 2H); 4.71 (t, J=8.5 Hz, 3H); 3.66 (dd, J=14.4, 7.4 Hz, 1H); 3.55 (d, J=6.9 Hz, 2H); 3.42 (dd, J=14.3, 6.0 Hz, 1H); 3.30 (q, J=6.2 Hz, 2H); 2.63-2.57 (m, 1H); 2.13-2.04 (m, 1H); 1.89 (dd, J=15.1, 9.8 Hz, 1H); 1.77-1.68 (m, 2H); 1.47 (s, 1H); 1.21 (d, J=6.2 Hz, 3H); 0.86 (d, J=5.4 Hz, 1H); 0.80 (q, J=7.6 Hz, 1H).

The following compounds of the present invention were made using the method described above and substituting the appropriate reactants and/or regents.

| | | LRMS |
|---|---|---|
| Compound | | (ESI) |
| No. | Structure | [M + 1]⁺ |
| 2 | 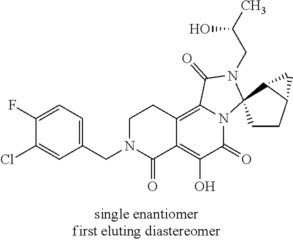 [SMILES: C[C@@H](O)CN1C(=O)c2c3c(c(O)c(=O)n2[C@@]12CC[C@H]1C[C@H]12)C(=O)N(Cc1ccc(F)c(Cl)c1)CC3] | 502 |
| 3 | 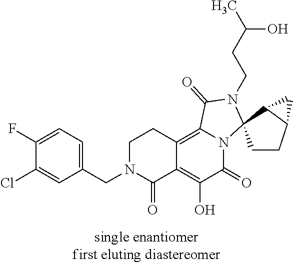 [SMILES: CC(O)CCN1C(=O)c2c3c(c(O)c(=O)n2[C@@]12CC[C@H]1C[C@H]12)C(=O)N(Cc1ccc(F)c(Cl)c1)CC3] | 516 |
| 4 | 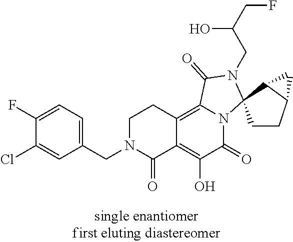 [SMILES: O=C1c2c(c3n(c(=O)c2O)[C@@]2(CC[C@H]4C[C@H]42)N(CC(O)CF)C3=O)CCN1Cc1ccc(F)c(Cl)c1] | 520 |
| 5 | 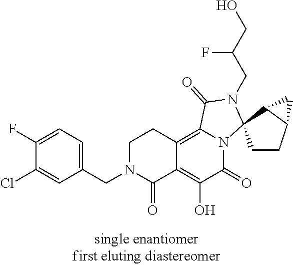 [SMILES: O=C1c2c(c3n(c(=O)c2O)[C@@]2(CC[C@H]4C[C@H]42)N(CC(F)CO)C3=O)CCN1Cc1ccc(F)c(Cl)c1] | 520 |
| 6 | 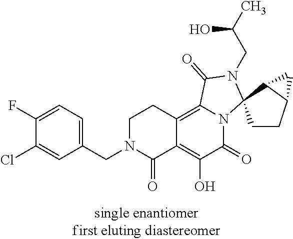 [SMILES: C[C@H](O)CN1C(=O)c2c3c(c(O)c(=O)n2[C@@]12CC[C@H]1C[C@H]12)C(=O)N(Cc1ccc(F)c(Cl)c1)CC3] | 502 |
| 7 | 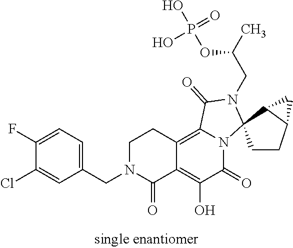 [SMILES: C[C@H](CN1C(=O)c2c3c(c(O)c(=O)n2[C@@]12CC[C@H]1C[C@H]12)C(=O)N(Cc1ccc(F)c(Cl)c1)CC3)OP(=O)(O)O] | 582 |

### Example 6

### Preparation of Compound 8

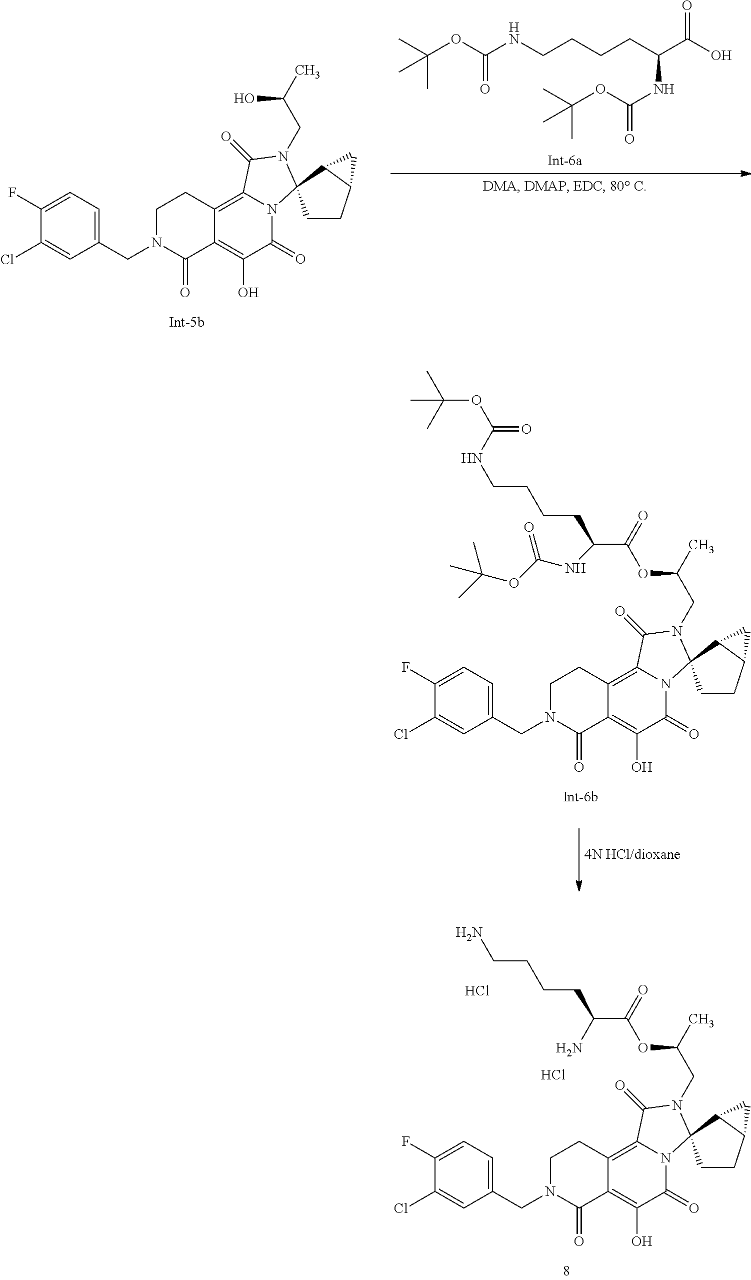 [SMILES: C[C@H](O)CN1C(=O)c2c3c(c(O)c(=O)n2[C@@]12CC[C@H]1C[C@H]12)C(=O)N(Cc1ccc(F)c(Cl)c1)CC3 ; CC(C)(C)OC(=O)NCCCC[C@H](NC(=O)OC(C)(C)C)C(=O)O ; C[C@@H](CN1C(=O)c2c3c(c(O)c(=O)n2[C@@]12CC[C@H]1C[C@H]12)C(=O)N(Cc1ccc(F)c(Cl)c1)CC3)OC(=O)[C@H](CCCCNC(=O)OC(C)(C)C)NC(=O)OC(C)(C)C ; C[C@@H](CN1C(=O)c2c3c(c(O)c(=O)n2[C@@]12CC[C@H]1C[C@H]12)C(=O)N(Cc1ccc(F)c(Cl)c1)CC3)OC(=O)[C@@H](N)CCCCN ; Cl ; Cl]

### Step A—Synthesis of Intermediate Compound Int-6b

To a DMA (19.9 mL) solution of compound Int-5b (2.0 g, 3.98 mmol, Example 1, Diastereomer A) was added DMAP (730 mg, 5.98 mmol) followed by EDC (3.06 g, 15.94 mmol). Solid BOC-LYS(BOC)-OH (Int-6a, 4.14 g, 11.95 mmol) was added and the reaction was heated to 80° C. and allowed to stir at this temperature until the reaction was complete by LCMS analysis (˜6 hours). The reaction was purified using gradient elution on reverse phase (SunFire™ Prep C18 OBD™ 10 um 50×250 mm column; 70-90% CH₃CN/water with 0.1% TFA modifier over 20 minutes) to provide compound Int-6b as a yellow solid (2.2 g, 65%). MS (+ESI) m/z=830.3. ¹H NMR (400 MHz, CDCl₃): δ 13.48 (s, 1H); 7.37 (dd, J=6.9, 2.2 Hz, 1H); 7.23-7.18 (m, 1H); 7.13 (t, J=8.6 Hz, 1H); 5.39-5.31 (m, 1H); 5.08 (s, 1H); 4.69 (s, 2H); 4.58 (s, 1H); 4.23 (s, 1H); 3.86 (dd, J=14.4, 7.8 Hz, 1H); 3.50-3.35 (m, 5H); 3.09 (t, J=6.7 Hz, 2H); 2.87 (dd, J=11.4, 5.5 Hz, 1H); 2.16-1.71 (m, 8H); 1.68-1.57 (m, 1H); 1.44 (s, 18H); 1.32 (d, J=6.3 Hz, 3H); 0.96 (q, J=7.3 Hz, 1H); 0.76 (s, 1H).

### Step B—Synthesis of Intermediate Compound 8

Compound Int-6b was taken up in 4N dioxane/HCl (19.92 ml, 80 mmol) and stirred at room temperature for 2 hours, then the reaction mixture was concentrated in vacuo. The residue obtained was triturated with diethyl ether then the ether was decanted off (3×50 mL). The yellow solid obtained was dried under high vacuum to provide compound 8 (2.0 g, 71%). MS (+ESI) m/z=630.3. ¹H NMR (400 MHz, DMSO): δ 13.53 (s, 1H); 8.59 (s, 3H); 7.99 (s, 3H); 7.58 (d, J=7.2 Hz, 1H); 7.45-7.35 (m, 2H); 5.35 (d, J=8.1 Hz, 1H); 4.72 (s, 2H); 3.97 (s, 1H); 3.77 (d, J=10.8 Hz, 3H); 3.38 (dd, J=14.0, 7.0 Hz, 1H); 3.29 (d, J=6.9 Hz, 2H); 2.74 (s, 2H); 2.64 (s, 1H); 2.13 (t, J=12.4 Hz, 1H); 1.96-1.74 (m, 3H); 1.66-1.42 (m, 3H); 1.27 (d, J=6.3 Hz, 3H); 1.12-1.05 (m, 1H); 0.89 (s, 1H); 0.77 (d, J=8.4 Hz, 1H).

The following compounds of the present invention were made using the method described above and substituting the appropriate reactants and/or regents.

| | | LRMS |
|---|---|---|
| Compound | | (ESI) |
| No. | Structure | [M + 1]⁺ |
| 9 | 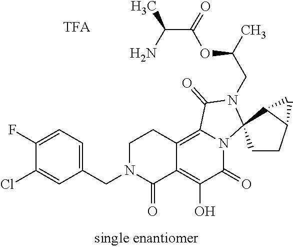 [SMILES: C[C@H](N)C(=O)O[C@@H](C)CN1C(=O)c2c3c(c(O)c(=O)n2[C@@]12CC[C@H]1C[C@H]12)C(=O)N(Cc1ccc(F)c(Cl)c1)CC3] | 574 |
| 10 | 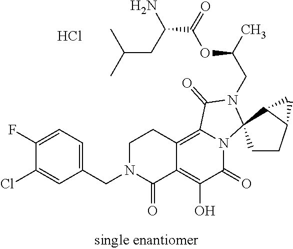 [SMILES: CC(C)C[C@H](N)C(=O)O[C@@H](C)CN1C(=O)c2c3c(c(O)c(=O)n2[C@@]12CC[C@H]1C[C@H]12)C(=O)N(Cc1ccc(F)c(Cl)c1)CC3 ; Cl] | 616 |
| 11 | 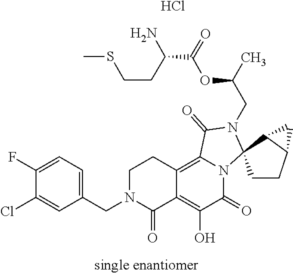 [SMILES: Cl ; CSCC[C@H](N)C(=O)O[C@@H](C)CN1C(=O)c2c3c(c(O)c(=O)n2[C@@]12CC[C@H]1C[C@H]12)C(=O)N(Cc1ccc(F)c(Cl)c1)CC3] | 634 |
| 12 | 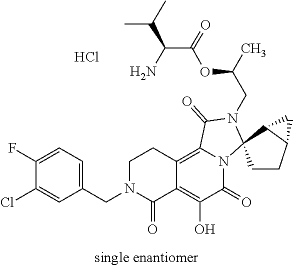 [SMILES: CC(C)[C@H](N)C(=O)O[C@@H](C)CN1C(=O)c2c3c(c(O)c(=O)n2[C@@]12CC[C@H]1C[C@H]12)C(=O)N(Cc1ccc(F)c(Cl)c1)CC3 ; Cl] | 602 |
| 13 | 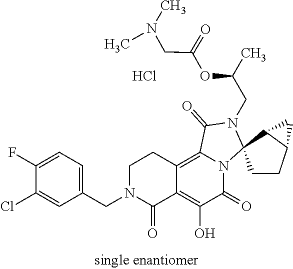 [SMILES: C[C@@H](CN1C(=O)c2c3c(c(O)c(=O)n2[C@@]12CC[C@H]1C[C@H]12)C(=O)N(Cc1ccc(F)c(Cl)c1)CC3)OC(=O)CN(C)C ; Cl] | 588 |

### Example 7

### Preparation of Compounds 14 and 15

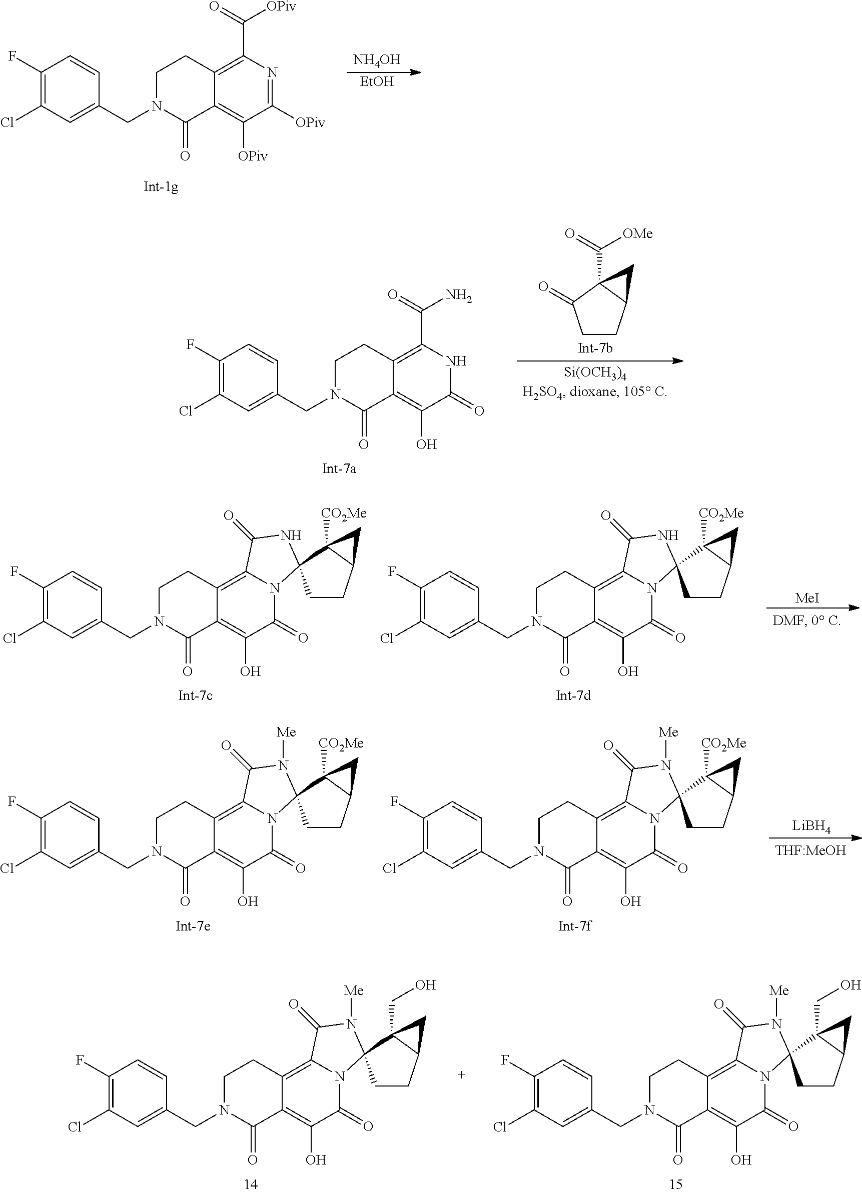

### Step A—Synthesis of Intermediate Compound Int-7a

To a suspension of Int-1g (85 mmol, 50 g) in ethanol (400 mL, 0.21 M) cooled in an ice bath, was added ammonium hydroxide (10 equiv, 28 wt %) over 30 minutes. The reaction was stirred at room temperature for 2 hours. The precipitate was filtered rinsing the solid with ethanol. The solid was stirred in THF and the mixture was acidified with 1 N HCl to pH=2-3. The resulting mixture was stirred for an hour and the solid was filtered and washed with THF to provide the primary amide as a white solid. (25.8 g, 70.5 mmol, 83%) LRMS (+ESI) m/z=366.1. ¹H NMR (400 MHz, DMSO): δ 13.2 (s, 1H); 11.4 (s, 1H); 7.7 (s, 2H); 7.60 (m, 1H); 7.44-7.34 (m, 2H); 4.70 (s, 2H); 3.51 (t, J=6.3 Hz, 2H); 3.35 (m, 2H).

### Step B—Synthesis of Intermediate Compounds Int-7c and Int-7d

A solution of 6-(3-chloro-4-fluorobenzyl)-4-hydroxy-3,5-dioxo-2,3,5,6,7,8-hexahydro-2,6-naphthyridine-1-carboxamide (from step 1) (2.25 g, 6.15 mmol), tetramethoxysilane (1.84 ml, 12.3 mmol) and (1R,5R)-methyl 2-oxobicyclo[3.1.0]hexane-1-carboxylate in dioxane (Int-7b, 20 mL, made racemically using the method described in Synlett 2007, 4, 579-582, then resolved using chiral SFC as follows: DAICEL CHIRALPAK IC-Column: 90% CO2-10% MeOH-0.1% DEA, t_{R}=3 2 min for enantiomer) was degassed with nitrogen for 10 min followed by addition of sulfuric acid (0.34 mL, 6.46 mmol). The mixture was capped in a pressure vessel and heated to 105° C. for 60 minutes. The mixture was cooled to room temperature and was partitioned between water (300 mL) and ethyl acetate (300 mL). The organic phase was washed with water (twice) and brine. The organic phase was dried over Na₂SO₄, filtered and concentrated in vacuo. The residue obtained was redissolved in ethyl acetate (10 mL) and diethyl ether (400 mL) and stirred overnight to form a white solid. The resulting white solid was collected by vacuum filtration, and washed with diethyl ether (2×250 mL). The solid was dried under high vacuum to provide a mixture of compound Int-7c and Int-7d as a white solid (2.35 g, 87%). LRMS (+ESI) m/z=502.3 found.

### Step C—Synthesis of Intermediate Compounds Int-7e and Int-7f

In a flame dried flask under N2, (1R,5R)-methyl 8′-(3-chloro-4-fluorobenzyl)-6′-hydroxy-1′,5′,7′-trioxo-2′,5′,7′,8′,9′,10′-hexahydro-1′H-spiro[bicyclo[3.1.0]hexane-2,3′-imidazo[5,1-a][2,6]naphthyridine]-1-carboxylate (2.75 g, 5.5 mmol) (mixture of C & D from step 2) was dissolved in anhydrous DMF (25 mL). The solution was degassed with nitrogen for 10 min, cooled in an ice bath and treated with sodium hydride (350 mg, 13.9 mmol). The mixture was stirred for 20 minutes at 0° C. and then treated with methyl iodide (0.55 mL, 8.8 mmol). The reaction was stirred for 20 minutes at 0° C. and then neutralized with 1N HCl. The mixture was diluted with EtOAc (500 mL) and then washed with water (2×) and brine (1×). The organic phase was dried over Na₂SO₄, filtered, and concentrated in vacuo. The resulting mixture of E and F was separated by gradient elution on reverse-phase (40-80% CH₃CN/water (0.1% TFA) over 35 min @ 85 mL/min on Sunfire Prep C18 50×250 mm). The earlier eluting diastereomer was collected and the acetonitrile was removed in vacuo. The pH was adjusted to 4-5 with dilute aq. NaHCO₃ and extracted with dichloromethane (3×). The combined organics were washed with brine, dried over Na₂SO₄, filtered, and concentrated in vacuo to provide compound F. (950 mg, 1.84 mmol, 33% yield). LRMS (+ESI) m/z=516.3 found. ¹H NMR (400 MHz, DMSO): δ 13.4 (s, 1H); 7.62 (d, J=7.2 Hz, 1H); 7.44-7.38 (m, 2H); 4.74 (d, J=15.0 Hz, 1H); 4.68 (d, J=15.0 Hz, 1H); 3.58 (t, J=6.2 Hz, 2H);); 3.37 (s, 3H); 3.35-3.29 (m, 2H); 3.01 (s, 3H); 2.71-2.61 (m, 1H); 2.40-2.28 (m, 2H); 1.98-1.90 (m, 1H); 1.77-1.71 (m, 1H); 1.65 (t, J=5.4 Hz, 1H); 1.61-1.56 (m, 1H).

### Step D—Synthesis of Compounds 14 and 15

In a flame dried flask under an atmosphere of nitrogen, (1R,2S,5R)-methyl 8′-(3-chloro-4-fluorobenzyl)-6′-hydroxy-2′-methyl-1′,5′,7′-trioxo-2′,5′,7′,8′,9′,10′-hexahydro-1′H-spiro[bicyclo[3.1.0]hexane-2,3′-imidazo[5,1-a][2,6]naphthyridine]-1-carboxylate (compound F) (460 mg, 0.89 mmol) was dissolved in anhydrous THF (25 mL). To this solution was added lithium chloride (148 mg, 3.9 mmol) and anhydrous methanol (0.76 mL, 18.7 mmol) and then it was cooled in an ice bath. Lithium borohydride (2.0M in THF, 3.1 mL, 6.2 mmol) was added dropwise over 5 minutes. After a few minutes, the ice bath was removed and stirred at room temperature. After 25 minutes the reaction was again cooled in an ice bath and recharged with the above amounts of methanol and then lithium borohydride. After a few minutes, the ice bath was removed and stirred at room temperature for another 20 minutes. This recharging was repeated twice more in the same interval. The reaction was then cooled in an ice bath and quenched with acetone (13 mL, 177 mmol). After 10 minutes stirring at room temperature, the reaction was diluted with dichloromethane and neutralized with aq. 1N HCl. The mixture was extracted with dichloromethane (3×) and the combined organics were dried over Na₂SO₄, filtered and concentrated in vacuo. The residue obtained was then purified using gradient elution on reverse-phase (35-75% CH₃CN/water (0.1% TFA) over 35 min @ 85 mL/min on Sunfire Prep C18 50×250 mm). The appropriate fractions were combined, the pH was adjusted to 4-5 with dilute aq. NaHCO₃, and then extracted with dichloromethane (3×). The combined organics were washed with brine, dried over Na₂SO₄, filtered, and concentrated in vacuo to provide the title compound. (227 mg, 0.47 mmol, 52% yield). LRMS (+ESI) m/z=488.3. ¹H NMR (400 MHz, DMSO): δ 13.45 (s, 1H); 7.60 (dd, J=7.2, 1.8 Hz, 1H); 7.42-7.37 (m, 2H); 4.71 (s, 2H); 4.34 (dd, J=7.0, 4.0 Hz, 1H); 3.56 (t, J=6.4 Hz, 2H); 3.40 (dd, J=11.9, 7.0 Hz, 1H); 3.32-3.25 (m, 2H); 3.02 (s, 3H); 2.89 (dd, J=11.9, 4.0 Hz, 1H); 2.69-2.60 (m. 1H); 2.25-2.18 (m, 1H); 1.96-1.92 (m, 1H); 1.67-1.62 (m, 1H); 1.58-1.52 (m, 1H); 0.98 (t, J=4.62 Hz, 1H); 0.93-0.87 (m, 1H).

### Example 8

### Preparation of Compounds 16 and 17

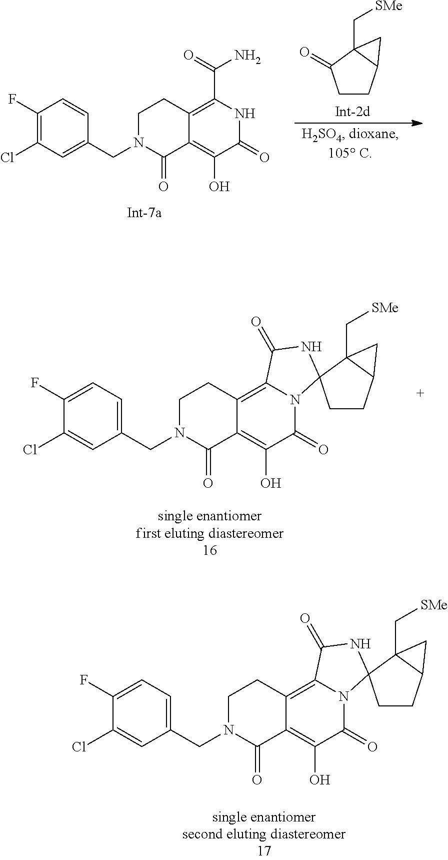 [SMILES: NC(=O)c1[nH]c(=O)c(O)c2c1CCN(Cc1ccc(F)c(Cl)c1)C2=O ; CSCC12CC1CCC2=O ; "H2SO4, dioxane, 105° C." ; CSCC12CC1CCC21NC(=O)c2c3c(c(O)c(=O)n21)C(=O)N(Cc1ccc(F)c(Cl)c1)CC3 ; CSCC12CC1CCC21NC(=O)c2c3c(c(O)c(=O)n21)C(=O)N(Cc1ccc(F)c(Cl)c1)CC3]

To a suspension of 6-(3-chloro-4-fluorobenzyl)-4-hydroxy-3,5-dioxo-2,3,5,6,7,8-hexahydro-2,6-naphthyridine-1-carboxamide (100 mg, 0.27 mmol) in dioxane (5 mL) was added 1-((methylthio)methyl)bicyclo[3.1.0]hexan-2-one (0.42 g, 2.7 mmol) (example 2, step D, Int-2d) and conc. H₂SO₄ (5 drops) at room temperature. The mixture was heated to 105° C. for 0.5 hour under microwave. After cooled to room temperature, the reaction was quenched with water (5 mL) and extracted with DCM (30 mL). The combined layers were dried over anhydrous MgSO₄, concentrated in vacuo and the residue obtained was purified using prep-HPLC to provide desired compound (20 mg, yield: 15%) as yellow solid. LRMS (+ESI) m/z=504.3 found, 504.4 required. ¹H NMR: (400 MHz, CDCl₃) δ 13.56 (brs, 1H), 7.85-8.12 (m, 1H), 7.38-7.41 (m, 1H), 7.21-7.24 (m, 1H), 7.13-7.17 (m, 1H), 4.63-4.75 (m, 2H), 2.79-3.53 (m, 6H), 1.31-2.40 (m, 9H), 0.69-0.89 (m, 1H). Compounds 16 and 17 were resolved using chiral SFC, requiring two successive purifications as follows: (1) DAICEL CHIRALCEL OD-Column: 60% CO₂-40% EtOH (0.05% NH₄OH), 45 mL/min, t_{R}=13.21 min for peak 1, t_{R}=23.15 min for peak 2, wherein each peak contained 2 enantiomers, then (2) peak 1 was separated into single enantiomers using a DAICEL CHIRALPAK AD-Column: 65% CO₂-35% EtOH (0.05% NH₄OH), 55 mL/min, t_{R}=11.09 min for compound 16 (first eluting compound from the peak 1 mixture), t_{R}=11.71 min for compound 17 (second eluting compound from the peak 1 mixture). Data for Isomers 16 and 17: LRMS (+ESI) m/z=504.2. ¹H NMR: (400 MHz, CDCl₃) δ 13.56 (brs, 1H), 8.07 (s, 1H), 7.38-7.40 (m, 1H), 7.21-7.24 (m, 1H), 7.12-7.17 (m, 1H), 4.59-4.73 (m, 2H), 3.32-3.61 (m, 4H), 2.72-2.89 (m, 2H), 2.34-2.40 (m, 1H), 2.15-2.24 (m, 1H), 2.01-2.06 (m, 1H), 1.98 (s, 3H), 1.77-1.84 (m, 2H), 0.88-0.92 (m, 1H), 0.70-0.73 (m, 1H).

### Example 9

### Preparation of Compound 18

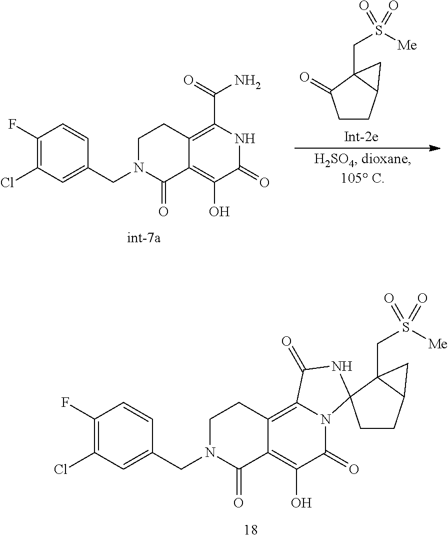 [SMILES: NC(=O)c1[nH]c(=O)c(O)c2c1CCN(Cc1ccc(F)c(Cl)c1)C2=O ; CS(=O)(=O)CC12CC1CCC2=O ; "H2SO4, dioxane, 105° C." ; CS(=O)(=O)CC12CC1CCC21NC(=O)c2c3c(c(O)c(=O)n21)C(=O)N(Cc1ccc(F)c(Cl)c1)CC3]

To a suspension of 1-((methylsulfonyl)methyl)bicyclo[3.1.0]hexan-2-one (100 mg, 0.27 mmol) in dioxane (5 mL) was added 1-((methylsulfonyl)methyl)bicycle [3.1.0]hexan-2-one (0.51 g, 2.7 mmol) and conc. H₂SO₄ (5 drops) at room temperature. The mixture was heated to 105° C. for 0.5 hour under microwave. After cooled to room temperature, the reaction was quenched with water (5 mL) and extracted with DCM (30 mL). The combined layers were dried over anhydrous MgSO₄, the solvent was removed in vacuo and the residue obtained was purified using prep-HPLC to provide compound 18 as a mixture of isomers (20 mg, yield: 14%) as a yellow solid. LRMS (+ESI) m/z=536.1. ¹H NMR: (400 MHz, CDCl₃) δ 13.61 (brs, 1H), 7.95-8.05 (m, 1H), 7.37-7.40 (m, 1H), 7.20-7.24 (m, 1H), 7.12-7.17 (m, 1H), 4.59-4.76 (m, 2H), 3.47-3.68 (m, 3H), 3.36-3.39 (m, 2H), 2.69-2.90 (m, 5H), 2.34-2.43 (m, 1H), 1.85-2.18 (m, 3H), 1.06-1.45 (m, 2H).

### Example 10

### Preparation of Compound 19

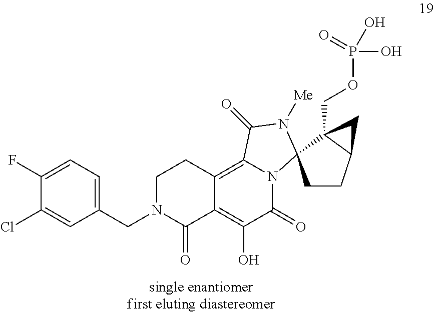 [SMILES: CN1C(=O)c2c3c(c(O)c(=O)n2[C@]12CC[C@@H]1C[C@@]12COP(=O)(O)O)C(=O)N(Cc1ccc(F)c(Cl)c1)CC3]

Compound 19 was made from compound 14 using the method described in Example 1, Step C. LRMS (+ESI) m/z=568.2. ¹H NMR (400 MHz, DMSO): δ 13.46 (s, 1H); 7.62-7.58 (m, 1H); 7.42-7.36 (m, 2H); 4.76-4.66 (m, 2H); 3.82 (dd, J=11.1, 7.1 Hz, 1H); 3.55 (t, J=6.5 Hz, 2H); 3.37-3.23 (m, 3H); 3.02 (s, 3H); 2.66-2.57 (m, 1H); 2.29-2.20 (m, 1H); 1.95 (dd, J=15.2, 9.5 Hz, 1H); 1.76-1.63 (m, 2H); 1.17-1.12 (m, 1H); 1.09-1.02 (m, 1H).

### Example 11

### Assay for Inhibition of HIV Replication

This assay is a kinetic assay that employs a reporter cell line (MT4-gag-GFP) to quantify the number of new cells infected in each round of replication. Briefly, MT4-GFP cells (250,000 cells/ml) were bulk-infected with HIV-1 (NL4-3 strain) at low multiplicity of infection (MOI) in RPMI+10% FBS for 24 hours. Cells were then washed once in RPMI+10% FBS and resuspended RPMI+10% or 50% normal human serum (NHS). Test compounds were serial-diluted in DMSO on ECHO. The infected MT4-GFP cells were added to a 384-well poly-D-lysine coated black plate with clear bottom in which the diluted test compounds were placed. The cells were seeded at 8,000 cells per well and the final DMSO concentration was 0.4%. The infected cells (Green GFP cells) were quantified at both 24 and 48 hours post incubation using Acumen eX3. Viral reproductive ratio (R₀) was determined using the number of infected cells at 48 hours divided by the number of infected cells at 24 hours. Percent viral growth inhibition was calculated by [1−(R−R_{tripledrug})/(R_{DMSO}−R_{tripledrug})]*100. Compound potency IP or IC50 was determined by a 4-parameter dose response curve analysis.

TABLE C

| | Wild Type Cell | |
|---|---|---|
| Compound No. | Assay IP (% NHS) | |
| 1 | 1200 | nM (50% NHS) |
| 2 | 33 | nM (50% NHS) |
| 3 | 32 | nM (50% NHS) |
| 4 | 7.5 | nM (10% NHS) |
| 5 | 27 | nM (10% NHS) |
| 6 | 53 | nM (50% NHS) |
| 7 | 1091 | nM (50% NHS) |
| 8 | 46 | nM (50% NHS) |
| 9 | 42 | nM (50% NHS) |
| 10 | 49 | nM (50% NHS) |
| 11 | 71 | nM (50% NHS) |
| 12 | 54 | nM (50% NHS) |
| 13 | 69 | nM (50% NHS) |
| 14 | 14 | nM (50% NHS) |
| 15 | 24 | nM (50% NHS) |
| 16 | 20 | nM (50% NHS) |
| 17 | 28 | nM (50% NHS) |
| 18 | 37 | nM (50% NHS) |
| 19 | 2086 | nM (50% NHS) |

### Uses of the Substituted Naphthyridinedione Derivatives

The Substituted Naphthyridinedione Derivatives are useful in human and veterinary medicine for treating or preventing HIV infection in a subject. In one embodiment, the Substituted Naphthyridinedione Derivatives can be inhibitors of HIV viral replication. In a specific embodiment, the Substituted Naphthyridinedione Derivatives are inhibitors of HIV-1. Accordingly, the Substituted Naphthyridinedione Derivatives are useful for treating HIV infections and AIDS. In accordance with the invention, the Substituted Naphthyridinedione Derivatives can be administered to a subject in need of treatment or prevention of HIV infection.

Accordingly, in one embodiment, the invention provides methods for treating HIV infection in a subject comprising administering to the subject an effective amount of at least one Substituted Naphthyridinedione Derivative or a pharmaceutically acceptable salt thereof. In a specific embodiment, the present invention provides methods for treating AIDS in a subject comprising administering to the subject an effective amount of at least one Substituted Naphthyridinedione Derivative or a pharmaceutically acceptable salt thereof

### Treatment or Prevention of HIV Infection

The Substituted Naphthyridinedione Derivatives are useful in the inhibition of HIV, the treatment of HIV infection and/or reduction of the likelihood or severity of symptoms of HIV infection and the inhibition of HIV viral replication and/or HIV viral production in a cell-based system. For example, the Substituted Naphthyridinedione Derivatives are useful in treating infection by HIV after suspected past exposure to HIV by such means as blood transfusion, exchange of body fluids, bites, accidental needle stick, or exposure to subject blood during surgery or other medical procedures.

In one embodiment, the HIV infection has progressed to AIDS.

Accordingly, in one embodiment, the invention provides methods for treating HIV infection in a subject, the methods comprising administering to the subject an effective amount of at least one Substituted Naphthyridinedione Derivative or a pharmaceutically acceptable salt thereof. In a specific embodiment, the amount administered is effective to treat or prevent infection by HIV in the subject. In another specific embodiment, the amount administered is effective to inhibit HIV viral replication and/or viral production in the subject.

The Substituted Naphthyridinedione Derivatives are also useful in the preparation and execution of screening assays for antiviral compounds. For example the Substituted Naphthyridinedione Derivatives are useful for identifying resistant HIV cell lines harboring mutations, which are excellent screening tools for more powerful antiviral compounds. Furthermore, the Substituted Naphthyridinedione Derivatives are useful in establishing or determining the binding site of other antivirals to the HIV Integrase.

The compositions and combinations of the present invention can be useful for treating a subject suffering from infection related to any HIV genotype.

### Combination Therapy

In another embodiment, the present methods for treating or preventing HIV infection can further comprise the administration of one or more additional therapeutic agents which are not Substituted Naphthyridinedione Derivatives.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a subject, the method comprising administering to the subject: (i) at least one Substituted Naphthyridinedione Derivative (which may include two or more different Substituted Naphthyridinedione Derivatives), or a pharmaceutically acceptable salt thereof, and (ii) at least one additional therapeutic agent that is other than a Substituted Naphthyridinedione Derivative, wherein the amounts administered are together effective to treat or prevent a viral infection.

When administering a combination therapy of the invention to a subject, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Substituted Naphthyridinedione Derivative and an additional therapeutic agent may be present in fixed amounts (dosage amounts) in a single dosage unit (e.g., a capsule, a tablet and the like).

In one embodiment, the at least one Substituted Naphthyridinedione Derivative is administered during a time when the additional therapeutic agent(s) exert their prophylactic or therapeutic effect, or vice versa.

In another embodiment, the at least one Substituted Naphthyridinedione Derivative and the additional therapeutic agent(s) are administered in doses commonly employed when such agents are used as monotherapy for treating a viral infection.

In another embodiment, the at least one Substituted Naphthyridinedione Derivative and the additional therapeutic agent(s) are administered in doses lower than the doses commonly employed when such agents are used as monotherapy for treating a viral infection.

In still another embodiment, the at least one Substituted Naphthyridinedione Derivative and the additional therapeutic agent(s) act synergistically and are administered in doses lower than the doses commonly employed when such agents are used as monotherapy for treating a viral infection.

In one embodiment, the at least one Substituted Naphthyridinedione Derivative and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HIV infection.

In another embodiment, the viral infection is AIDS.

The at least one Substituted Naphthyridinedione Derivative and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Substituted Naphthyridinedione Derivative and the additional therapeutic agent(s) may inhibit the resistance of a viral infection to these agents.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| CMX-157 | nRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| Dolutegravir | PI |
| efavirenz, EFV, Sustiva ®, Stocrin ® | nnRTI |
| efavirenz + emtricitabine + tenofovir DF, Atripla ® | nnRTI + |
| | nRTI |
| Elvitegravir | InI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| raltegravir, MK-0518, Isentress ® | InI |
| rilpivirine, TMC-278 | nnRTI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| tipranavir, Aptivus ® | PI |

EI = entry inhibitor;
FI = fusion inhibitor;
InI = integrase inhibitor;
PI = protease inhibitor;
nRTI = nucleoside reverse transcriptase inhibitor;
nnRTI = non-nucleoside reverse transcriptase inhibitor.
Some of the drugs listed in the table are used in a salt form; e.g., abacavir sulfate, indinavir sulfate, atazanavir sulfate, nelfinavir mesylate.

In one embodiment, the one or more anti-HIV drugs are selected from raltegravir, lamivudine, abacavir, ritonavir, dolutegravir, darunavir, atazanavir, emtricitabine, tenofovir, elvitegravir, rilpivirine and lopinavir.

In another embodiment, the compound of formula (I) is used in combination with a single anti-HIV drug which is raltegravir.

In another embodiment, the compound of formula (I) is used in combination with a single anti-HIV drug which is lamivudine.

In still another embodiment, the compound of formula (I) is used in combination with a single anti-HIV drug which is atazanavir.

In another embodiment, the compound of formula (I) is used in combination with a single anti-HIV drug which is darunavir.

In another embodiment, the compound of formula (I) is used in combination with a single anti-HIV drug which is rilpivirine.

In yet another embodiment, the compound of formula (I) is used in combination with a single anti-HIV drug which is dolutegravir.

In another embodiment, the compound of formula (I) is used in combination with a single anti-HIV drug which is elvitegravir.

In one embodiment, the compound of formula (I) is used in combination with two anti-HIV drugs which are lamivudine and abacavir.

In another embodiment, the compound of formula (I) is used in combination with two anti-HIV drugs which are darunavir and raltegravir.

In another embodiment, the compound of formula (I) is used in combination with two anti-HIV drugs which are emtricitabine and tenofovir.

In still another embodiment, the compound of formula (I) is used in combination with two anti-HIV drugs which are atazanavir and raltegravir.

In another embodiment, the compound of formula (I) is used in combination with two anti-HIV drugs which are ritonavir and lopinavir.

In another embodiment, the compound of formula (I) is used in combination with two anti-HIV drugs which are lamivudine and raltegravir.

In one embodiment, the compound of formula (I) is used in combination with three anti-HIV drug which are abacavir, lamivudine and raltegravir.

In another embodiment, the compound of formula (I) is used in combination with three anti-HIV drug which are lopinavir, ritonavir and raltegravir.

In one embodiment, the present invention provides pharmaceutical compositions comprising (i) a compound of formula (I) or a pharmaceutically acceptable salt thereof; (ii) a pharmaceutically acceptable carrier; and (iii) one or more additional anti-HIV agents selected from lamivudine, abacavir, ritonavir and lopinavir, or a pharmaceutically acceptable salt thereof, wherein the amounts present of components (i) and (iii) are together effective for the treatment or prophylaxis of infection by HIV or for the treatment, prophylaxis, or delay in the onset or progression of AIDS in the subject in need thereof.

In another embodiment, the present invention provides a method for the treatment or prophylaxis of infection by HIV or for the treatment, prophylaxis, or delay in the onset or progression of AIDS in a subject in need thereof, which comprises administering to the subject (i) a compound of formula (I) or a pharmaceutically acceptable salt thereof and (ii) one or more additional anti-HIV agents selected from lamivudine, abacavir, ritonavir and lopinavir, or a pharmaceutically acceptable salt thereof, wherein the amounts administered of components (i) and (ii) are together effective for the treatment or prophylaxis of infection by HIV or for the treatment, prophylaxis, or delay in the onset or progression of AIDS in the subject in need thereof

It is understood that the scope of combinations of the compounds of this invention with anti-HIV agents is not limited to the HIV antivirals listed in Table A, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), the 59^{th} edition (2005), and the like. The dosage ranges for a compound of the invention in these combinations are the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

The doses and dosage regimen of the other agents used in the combination therapies of the present invention for the treatment or prevention of HIV infection can be determined by the attending clinician, taking into consideration the approved doses and dosage regimen in the package insert; the age, sex and general health of the subject; and the type and severity of the viral infection or related disease or disorder. When administered in combination, the Substituted Naphthyridinedione Derivative(s) and the other agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the pharmaceutical compositions are different, e.g., one is a tablet and one is a capsule. A kit comprising the separate dosage forms is therefore advantageous.

### Compositions and Administration

When administered to a subject, the Substituted Naphthyridinedione Derivatives can be administered as a component of a composition that comprises a pharmaceutically acceptable carrier or vehicle. The present invention provides pharmaceutical compositions comprising an effective amount of at least one Substituted Naphthyridinedione Derivative and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Substituted Naphthyridinedione Derivatives are administered orally.

In another embodiment, the one or more Substituted Naphthyridinedione Derivatives are administered intravenously.

In one embodiment, a pharmaceutical preparation comprising at least one Substituted Naphthyridinedione Derivative is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Substituted Naphthyridinedione Derivative(s) by weight or volume. In various embodiments, the present compositions can contain, in one embodiment, from about 1% to about 70% or from about 5% to about 60% of the Substituted Naphthyridinedione Derivative(s) by weight or volume.

The compounds of Formula I can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the subject to be treated. The specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Substituted Naphthyridinedione Derivatives will be regulated according to the judgment of the attending clinician considering such factors as age, condition and size of the subject as well as severity of the symptoms being treated. The compositions of the invention can further comprise one or more additional therapeutic agents, selected from those listed above herein. Accordingly, in one embodiment, the present invention provides compositions comprising: (i) at least one Substituted Naphthyridinedione Derivative or a pharmaceutically acceptable salt thereof; (ii) one or more additional therapeutic agents that are not a Substituted Naphthyridinedione Derivative; and (iii) a pharmaceutically acceptable carrier, wherein the amounts in the composition are together effective to treat HIV infection.

### Kits

In one aspect, the present invention provides a kit comprising a therapeutically effective amount of at least one Substituted Naphthyridinedione Derivative, or a pharmaceutically acceptable salt or prodrug of said compound and a pharmaceutically acceptable carrier, vehicle or diluent.

In another aspect the present invention provides a kit comprising an amount of at least one Substituted Naphthyridinedione Derivative, or a pharmaceutically acceptable salt or prodrug of said compound and an amount of at least one additional therapeutic agent listed above, wherein the amounts of the two or more active ingredients result in a desired therapeutic effect. In one embodiment, the one or more Substituted Naphthyridinedione Derivatives and the one or more additional therapeutic agents are provided in the same container. In one embodiment, the one or more Substituted Naphthyridinedione Derivatives and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 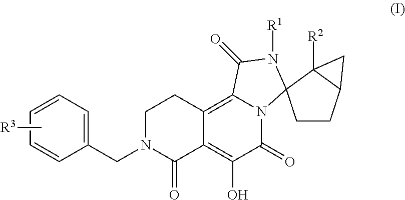
or a pharmaceutically acceptable salt thereof,
wherein:
R¹ is H or C₁-C₄ alkyl, wherein said C₁-C₄ alkyl group can be optionally substituted with up to two groups, each independently selected from —OH, F, —OP(O)(OH)₂ and —OC(O)(C₁-C₆ alkyl), wherein the C₁-C₆ alkyl moiety of said —OC(O)(C₁-C₆ alkyl) substituent group can be optionally substituted with up to 2 groups, each independently selected from
—N(R⁵)₂, —C(O)N(R⁵)₂ and —S—(C₁-C₃ alkyl);
R² is H or —(C₁-C₃ alkylene)-R⁴;
R³ represents up to 2 optional phenyl ring substituents, which are each independently selected from halo;
R⁴ is selected from —OH, —SH, —S—(C₁-C₃ alkyl), —SO₂(C₁-C₃ alkyl) and —OP(O)(OH)₂; and
each occurrence of R⁵ is H or C₁-C₆ alkyl, or two R⁵ groups that are attached to the same nitrogen atom, together with the common nitrogen atom to which they are attached, join to form a 4 to 7-membered heterocycloalkyl group.

2. The compound of claim 1, having the formula: 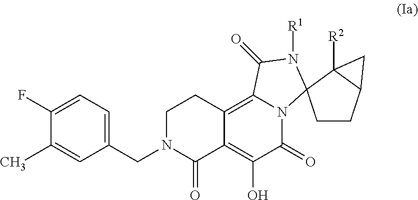
or a pharmaceutically acceptable salt thereof,
wherein:
R¹ is H or C₁-C₄ alkyl, wherein said C₁-C₄ alkyl group can be optionally substituted with up to two groups, each independently selected from —OH, F, —OP(O)(OH)₂ and —OC(O)(C₁-C₆ alkyl), wherein the C₁-C₆ alkyl moiety of said —OC(O)(C₁-C₆ alkyl) substituent group can be optionally substituted with up to 2 groups, each independently selected from —NH₂, —N(CH₃)₂, —C(O)NH₂ and —SCH₃;
R² is H or —CH₂R⁴; and
R⁴ is selected from —OH, —SCH₃, —SO₂CH₃ and —OP(O)(OH)₂.

3. The compound of claim 1, wherein R¹ is H.

4. The compound of claim 1, wherein R¹ is methyl.

5. The compound of claim 1, wherein R¹ is C₁-C₄ alkyl, which can be optionally substituted as set forth in claim 1.

6. The compound of claim 1, wherein R² is H.

7. The compound of claim 1, wherein R² is —(C₁-C₃ alkylene)-R⁴.

8. The compound of claim 1, wherein R¹ is selected from H, methyl, —CH₂CH(OH)CH₃, —CH₂CH₂CH(OH)CH₃, —CH₂CH(OH)CH₂F, —CH₂CH(F)CH₂OH, —CH₂CH(—OP(O)(OH)₂)CH₃, —OC(O)CH(NH₂)CH₂CH(CH₃)₂, —OC(O)CH(CH₃)—NH₂, —OC(O)CH(NH₂)CH₂CH₂C(O)NH₂, —OC(O)CH(NH₂)CH₂CH₂SCH₃, —OC(O)CH(isopropyl)-NH₂ and —OC(O)CH₂N(CH₃)₂.

9. The compound claim 8, wherein R² is selected from H, —CH₂OH, —CH₂SCH₃, —CH₂SO₂CH₃ and —CH₂OP(O)(OH)₂.

10. The compound of claim 1 having the structure: 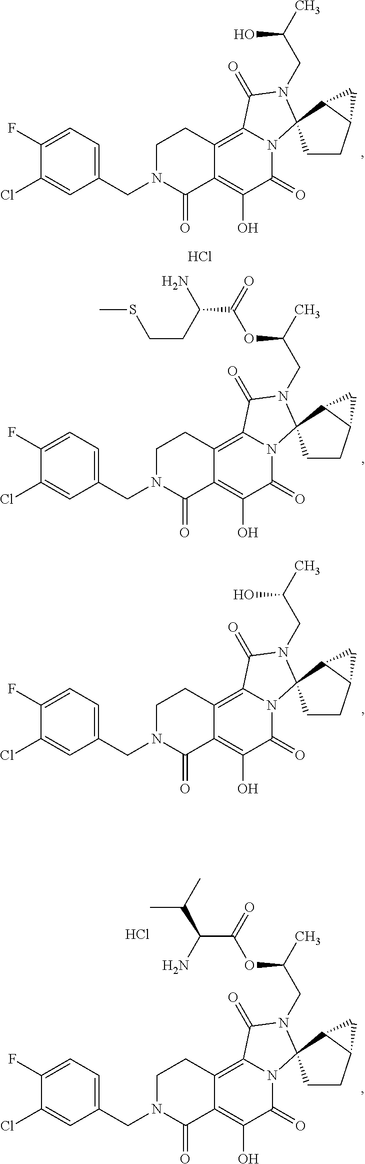 [SMILES: C[C@H](O)CN1C(=O)c2c3c(c(O)c(=O)n2[C@@]12CC[C@H]1C[C@H]12)C(=O)N(Cc1ccc(F)c(Cl)c1)CC3 ; Cl ; CSCC[C@H](N)C(=O)O[C@@H](C)CN1C(=O)c2c3c(c(O)c(=O)n2[C@@]12CC[C@H]1C[C@H]12)C(=O)N(Cc1ccc(F)c(Cl)c1)CC3 ; C[C@@H](O)CN1C(=O)c2c3c(c(O)c(=O)n2[C@@]12CC[C@H]1C[C@H]12)C(=O)N(Cc1ccc(F)c(Cl)c1)CC3 ; CC(C)[C@H](N)C(=O)O[C@@H](C)CN1C(=O)c2c3c(c(O)c(=O)n2[C@@]12CC[C@H]1C[C@H]12)C(=O)N(Cc1ccc(F)c(Cl)c1)CC3 ; Cl] 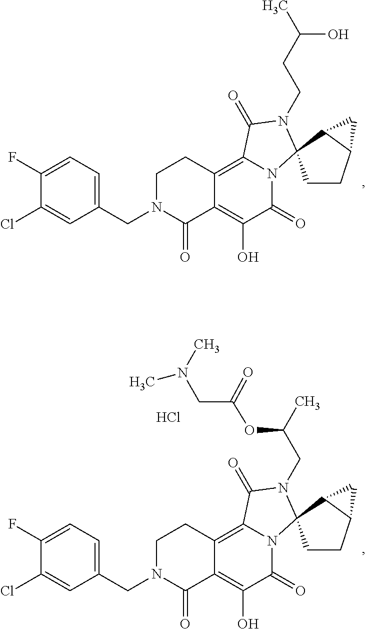 [SMILES: CC(O)CCN1C(=O)c2c3c(c(O)c(=O)n2[C@@]12CC[C@H]1C[C@H]12)C(=O)N(Cc1ccc(F)c(Cl)c1)CC3 ; C[C@@H](CN1C(=O)c2c3c(c(O)c(=O)n2[C@@]12CC[C@H]1C[C@H]12)C(=O)N(Cc1ccc(F)c(Cl)c1)CC3)OC(=O)CN(C)C ; Cl] 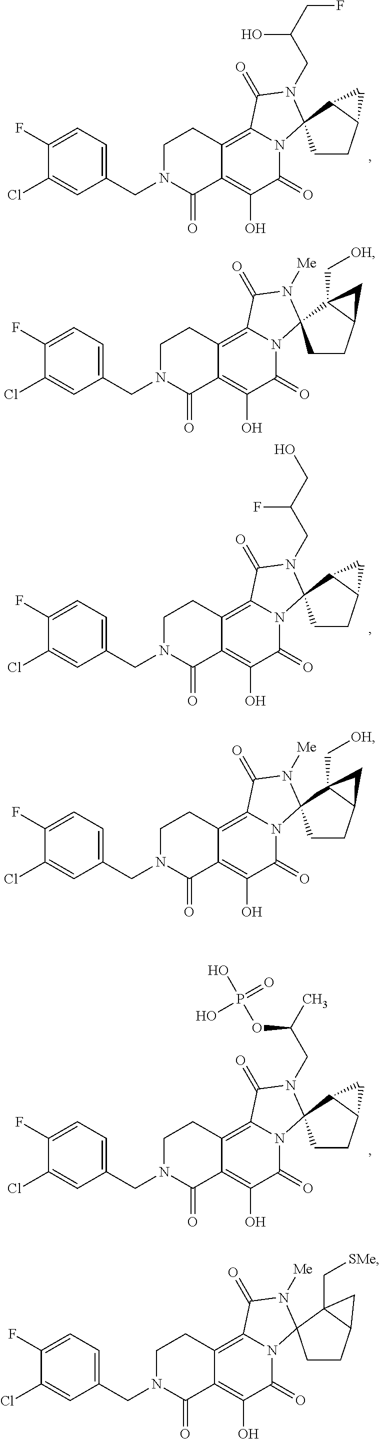 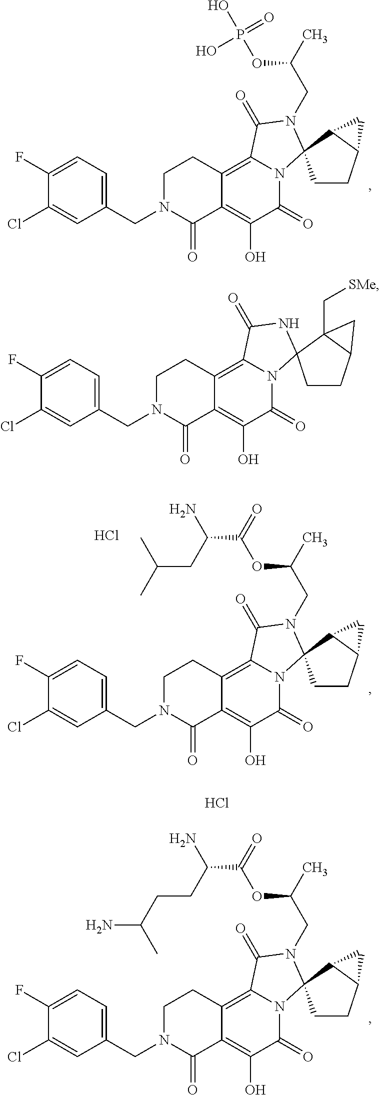 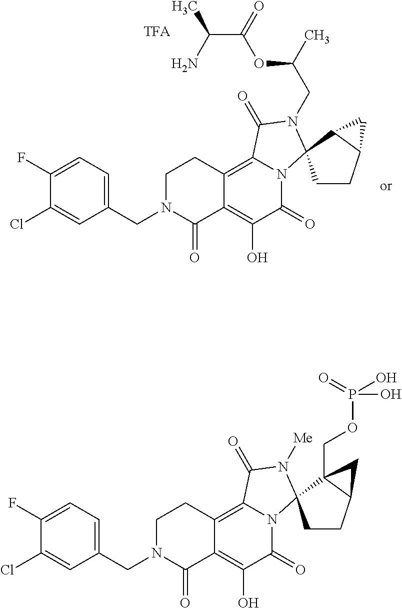 [SMILES: C[C@H](N)C(=O)O[C@@H](C)CN1C(=O)c2c3c(c(O)c(=O)n2[C@@]12CC[C@H]1C[C@H]12)C(=O)N(Cc1ccc(F)c(Cl)c1)CC3 ; CN1C(=O)c2c3c(c(O)c(=O)n2[C@]12CC[C@@H]1C[C@@]12COP(=O)(O)O)C(=O)N(Cc1ccc(F)c(Cl)c1)CC3]
or a pharmaceutically acceptable salt thereof.

11. A pharmaceutical composition comprising an effective amount of a compound according to claim 1, or a pharmaceutically acceptable salt thereof, and a pharmaceutically acceptable carrier.

12. A method for the inhibition of HIV integrase in a subject in need thereof which comprises administering to the subject an effective amount of the compound according to claim 1, or a pharmaceutically acceptable salt thereof.

13. A method for the treatment of infection by HIV or for the treatment or delay in the onset or progression of AIDS in a subject in need thereof, which comprises administering to the subject an effective amount of the compound according to claim 1, or a pharmaceutically acceptable salt thereof.

14. (canceled)

15. (canceled)

16. The pharmaceutical composition of claim 11, further comprising one or more additional therapeutic agents selected from raltegravir, lamivudine, abacavir, ritonavir, dolutegravir, arunavir, atazanavir, emtricitabine, tenofovir, elvitegravir, rilpivirine and lopinavir.

17. The method of claim 13, further comprising administering to the subject one or more additional therapeutic agents selected from raltegravir, abacavir, lamivudine, ritonavir and lopinavir, wherein the amounts administered of the compound of claim 1 and the one or more additional therapeutic agents, are together effective to treat infection by HIV or to treat or delay the onset or progression of AIDS.
